SEQUENCE LISTING (45 sequences)

SEQ ID NO: 1 - DNA, 831 bp, synthetic construct
  source 1..831: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgctctagattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 2 - DNA, 951 bp, synthetic construct
  source 1..951: mol_type = other DNA
atgggtaccagtctcctatgctgggtggtcctgggtttcctagggacagattctgtttccacagatcacacaggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgctgtgccagcagctcgcacctggacggatataatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 3 - protein, 276 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCALDSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 116 aa, synthetic construct
AQTVTQSQPEMSVQEAETVTLSCTYDTSESDYYLFWYKQPPSRQMILVIRQEAYKQQNATENRFSVNFQKAAKSFSLKISDSQLGDAAMYFCALDSGGGADGLTFGKGTHLIIQPY

SEQ ID NO: 5 - protein, 20 aa, synthetic construct
MACPGFLWALVISTCLEFSM

SEQ ID NO: 6 - protein, 7 aa, synthetic construct
TSESDYY

SEQ ID NO: 7 - protein, 8 aa, synthetic construct
QEAYKQQN

SEQ ID NO: 8 - protein, 12 aa, synthetic construct
ALDSGGGADGLT

SEQ ID NO: 9 - protein, 316 aa, synthetic construct
MGTSLLCWVVLGFLGTDSVSTDHTGAGVSQSPRYKVTKRGQDVTLRCDPISSHATLYWYQQALGQGPEFLTYFNYEAQPDKSGLPSDRFSAERPEGSISTLTIQRTEQRDSAMYRCASSSHLDGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 10 - protein, 116 aa, synthetic construct
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSSHLDGYNEQFFGPGTRLTVLE

SEQ ID NO: 11 - protein, 24 aa, synthetic construct
MGTSLLCWVVLGFLGTDSVSTDHT

SEQ ID NO: 12 - protein, 5 aa, synthetic construct
SSHAT

SEQ ID NO: 13 - protein, 6 aa, synthetic construct
FNYEAQ

SEQ ID NO: 14 - protein, 13 aa, synthetic construct
ASSSHLDGYNEQF

SEQ ID NO: 15 - protein, 9 aa, synthetic construct
SQYPSLWSV

SEQ ID NO: 16 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 176 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 18 - protein, 5 aa, synthetic construct
GSGGS

SEQ ID NO: 19 - protein, 4 aa, synthetic construct
GGGS

SEQ ID NO: 20 - protein, 4 aa, synthetic construct
GGSG

SEQ ID NO: 21 - protein, 5 aa, synthetic construct
GGSGG

SEQ ID NO: 22 - protein, 5 aa, synthetic construct
GSGSG

SEQ ID NO: 23 - protein, 5 aa, synthetic construct
GSGGG

SEQ ID NO: 24 - protein, 5 aa, synthetic construct
GGGSG

SEQ ID NO: 25 - protein, 5 aa, synthetic construct
GSSSG

SEQ ID NO: 26 - protein, 14 aa, synthetic construct
CALDSGGGADGLTF

SEQ ID NO: 27 - protein, 15 aa, synthetic construct
CASSSHLDGYNEQFF

SEQ ID NO: 28 - DNA, 807 bp, synthetic construct
  source 1..807: mol_type = other DNA
atgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgtggacgacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 29 - DNA, 924 bp, synthetic construct
  source 1..924: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgtggagattgggacggccaacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 30 - protein, 268 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDDNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 110 aa, synthetic construct
ELKVEQNPLFLSMQEGKNYTIYCNYSTTSDRLYWYRQDPGKSLESLFVLLSNGAVKQEGRLMASLDTKARLSTLHITAAVHDLSATYFCAVDDNARLMFGDGTQLVVKPN

SEQ ID NO: 32 - protein, 18 aa, synthetic construct
MKKLLAMILWLQLDRLSG

SEQ ID NO: 33 - protein, 5 aa, synthetic construct
TTSDR

SEQ ID NO: 34 - protein, 7 aa, synthetic construct
LLSNGAV

SEQ ID NO: 35 - protein, 9 aa, synthetic construct
AVDDNARLM

SEQ ID NO: 36 - protein, 307 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEIGTANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 37 - protein, 115 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEIGTANNEQFFGPGTRLTVL

SEQ ID NO: 38 - protein, 15 aa, synthetic construct
MLSLLLLLLGLGSVF

SEQ ID NO: 39 - protein, 5 aa, synthetic construct
SQVTM

SEQ ID NO: 40 - protein, 7 aa, synthetic construct
ANQGSEA

SEQ ID NO: 41 - protein, 12 aa, synthetic construct
SVEIGTANNEQF

SEQ ID NO: 42 - protein, 140 aa, synthetic construct; identical to SEQ ID NO: 16

SEQ ID NO: 43 - protein, 177 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 44 - protein, 11 aa, synthetic construct
CAVDDNARLMF

SEQ ID NO: 45 - protein, 14 aa, synthetic construct
CSVEIGTANNEQFF

(19) United States
(12) Patent Application Publication
(10) Pub. No.: US 2026/0356910 A1
(43) Pub. Date: Nov. 12, 2026

(54) PEPTIDES AND ENGINEERED T CELL RECEPTORS TARGETING BRACHYURY ANTIGEN AND METHODS OF USE

(71) Applicant: BOARD OF REGENTS, THE UNIVERSITY OF TEXAS SYSTEM, Austin, TX (US)

(72) Inventors: Ke PAN, Houston, TX (US); Cassian YEE, Houston, TX (US); Yulun CHIU, Houston, TX (US)

(73) Assignee: BOARD OF REGENTS, THE UNIVERSITY OF TEXAS SYSTEM, Austin, TX (US)

(21) Appl. No.: 19/486,842

(22) PCT Filed: May 21, 2024
(86) PCT No.: PCT/US2024/030400

§ 371 Date: Nov. 21, 2025

Related U.S. Application Data
(60) Provisional application No. 63/468,208, filed on May 22, 2023.
(60) Provisional application No. 63/545,670, filed on Oct. 25, 2023.

(51) Int. Cl. C07K 14/705; A61K 40/11; A61K 40/32; A61K 40/42; C07K 16/28; C07K 16/46

(52) U.S. Cl. CPC C07K 14/7051; A61K 40/11; A61K 40/32; A61K 40/42; C07K 16/2809; C07K 16/46; A61K 2239/55; C07K 2317/565; C07K 2319/02

(57) ABSTRACT

This disclosure provides for engineered T cell Receptors (TCRs), cells comprising the TCRs, and methods of making and using the TCRs. The current disclosure relates to TCRs that specifically recognize epitope(s) from tumor antigen TBXT. Accordingly, the disclosure relates to engineered T-cell Receptors (TCRs), nucleic acids encoding the TCRs, and cells comprising the nucleic acids and TCRs. Also provided are compositions comprising the cells, nucleic acids, or engineered TCRs of the disclosure, methods of making the cells and methods of using the embodiments of the disclosure for therapeutic treatments.

This application claims priority of U.S. Provisional Patent Application No. 63/468,208, filed May 22, 2023 and U.S. Provisional Patent Application No. 63/545,670, filed Oct. 25, 2023, which are hereby incorporated by reference in their entirety.

The application contains a Sequence Listing in compliance with ST.26 format and is hereby incorporated by reference in its entirety. Said Sequence Listing, created on May 20, 2024 is named MDACP1364WO.xml and is 45,645 bytes in size.

### BACKGROUND OF THE INVENTION

### I. Field of the Invention

This invention relates to the field of cancer therapy.

### II. Background

Adoptive T-cell therapy is one potentially powerful treatment for cancer that genetically modifies natural T cells to make them tumor-specific and to improve their ability to destroy tumor cells. The genetically modified T cells are able to express chimeric antigen receptors (CARs) or T-cell receptors (TCRs), showing impressive results in multiple clinical trials. TCR-engineered T (TCR-T) cells have shown great promise against tumors. The potency of TCRs relies on their interaction with peptide-major histocompatibility complex (pMHC), complexes formed by peptide bound to MHC. Intracellular antigens are cut up into peptide chains and displayed by MHC molecules to form pMHCs. Cytoplasmic proteins to be expressed by class I MHC proteins, most of which are defective ribosomal translation products, are cleaved into peptide chains by proteolysis. These peptides are then bound to class I MHC proteins, which are expressed on all nucleated cells' cell surface. Some cells, called antigen-presenting cells (APCs), express class II MHC proteins. They internalize foreign material proteins by endocytosis and cleave them into peptide chains to bind with class II MHC proteins T-cell receptors from T cells, which must be matched to human leukocyte antigen (HLA) alleles of patients, recognize these pMHCs and cause the killing of cancer cells. (Human class I MHC protein is expressed from 3 gene regions: HLA-A, HLA-B, and HLA-C, and human class II MHC protein is also expressed from 3 gene regions: HLA-DR, HLA-DP, and HLA-DQ.) There is a need for the engineering of TCRs that are directed to cancer-specific antigens and useful for the treatment of cancer.

### SUMMARY OF THE INVENTION

This disclosure provides for peptides useful for vaccination and other applications, engineered T cell Receptors (TCRs), cells comprising the peptides and TCRs, and methods of making and using the peptides and TCRs. The current disclosure relates to TCRs that specifically recognize TBXT (Brachyury) antigen, such as a peptide having the amino acid sequence of SQYPSLWSV (SEQ ID NO: 15).

The disclosure describes polypeptides comprising an antigen binding variable region comprising a CDR3 comprising an amino acid sequence with at least 80% sequence identity to SEQ ID NO: 8. Also described are polypeptides comprising an antigen binding variable region comprising a CDR3 comprising an amino acid sequence with at least 80% sequence identity to SEQ ID NO: 14.

The disclosure describes polypeptides comprising an antigen binding variable region comprising a CDR3 comprising an amino acid sequence with at least 80% sequence identity to SEQ ID NO: 35. Also described are polypeptides comprising an antigen binding variable region comprising a CDR3 comprising an amino acid sequence with at least 80% sequence identity to SEQ ID NO: 41.

The disclosure also provides for T-cell receptors (TCR) and engineered TCRs, such as a TCR comprising a TCR-a polypeptide and a TCR-b polypeptide, wherein the TCR-a polypeptide comprises a CDR3 comprising an amino acid sequence with at least 80% sequence identity to SEQ ID NO: 8 and the TCR-b polypeptide comprises a CDR3 comprising an amino acid sequence with at least 80% sequence identity to SEQ ID NO: 14. The TCR-a polypeptide may comprise a CDR3 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 8 and the TCR-b polypeptide may comprise a CDR3 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 14. The TCR-a polypeptide may comprise a CDR3 comprising the amino acid sequence of SEQ ID NO: 8 and the TCR-b polypeptide may comprise a CDR3 comprising the amino acid sequence of SEQ ID NO: 14.

The disclosure also provides for T-cell receptors (TCR) and engineered TCRs, such as a TCR comprising a TCR-a polypeptide and a TCR-b polypeptide, wherein the TCR-a polypeptide comprises a CDR3 comprising an amino acid sequence with at least 80% sequence identity to SEQ ID NO: 35 and the TCR-b polypeptide comprises a CDR3 comprising an amino acid sequence with at least 80% sequence identity to SEQ ID NO: 41. The TCR-a polypeptide may comprise a CDR3 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 35 and the TCR-b polypeptide may comprise a CDR3 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 41. The TCR-a polypeptide may comprise a CDR3 comprising the amino acid sequence of SEQ ID NO: 35 and the TCR-b polypeptide may comprise a CDR3 comprising the amino acid sequence of SEQ ID NO: 41.

The disclosure provides for a fusion protein comprising a TCR of the disclosure and a CD3 binding region. The CD3 binding region may comprise a CD3-specific fragment antigen binding (Fab), single chain variable fragment (scFv), single domain antibody, or single chain antibody. Exemplary CD3-specific fragment antigen binding (Fab) are known in the art. For example, US20180222981, which is herein incorporated by reference, discloses variable regions that bind specifically to CD3, which may be used in methods, compositions, cells, and polypeptides of this disclosure. Anti-CD3 antibodies and variable regions are disclosed in US20180117152, which is also incorporated by reference. A CD3 binding region may be excluded from the polypeptides and TCRs of the disclosure.

Also provided is a peptide comprising at least 66% sequence identity to a peptide of SEQ ID NO: 15. The peptide may be less than 100 amino acids in length. Also disclosed are polypeptides comprising a peptide of the disclosure. The peptide or polypeptide may have or have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 15. The peptide may consist of a peptide having the length and amino acid sequence of SEQ ID NO: 15.

Also described is a molecular complex comprising the peptide or polypeptide of the disclosure and a MHC polypeptide. The disclosure relates to a method of producing peptide-specific immune effector cells comprising: contacting a starting population of immune effector cells with a peptide of the disclosure, thereby generating peptide-specific immune effector cells. The disclosure also describes peptide-specific engineered T cells and TCRs produced according to the methods of the disclosure. Also provided is an in vitro isolated dendritic cell comprising a peptide, polypeptide, nucleic acid, or expression vector of the disclosure.

Also provided is a method for prognosing a patient or for detecting T cell responses in a patient, the method comprising: contacting a biological sample from the patient with a composition, peptide, or polypeptide of the disclosure. Also described is a peptide-specific binding molecule that bind to a peptide of the disclosure or that bind to a peptide-MHC complex. Exemplary binding molecules include antibodies, TCR mimic antibodies, scFvs, nanobodies, aptamers, and DARPINs. Binding molecules may exclude antibodies, TCR mimic antibodies, scFvs, nanobodies, aptamers, or DARPINs. Related methods provide for a method comprising contacting a composition comprising at least one MHC polypeptide and a peptide or polypeptide of the disclosure with a composition comprising T cells and detecting T cells with bound peptide and/or MHC polypeptide by detecting a detection tag.

Also described are kits comprising a peptide, polypeptide, nucleic acid, expression vector, or composition of the disclosure. The disclosure provides for a method of cloning a T cell receptor (TCR), the method comprising (a) contacting a starting population of immune effector cells with the peptide of the disclosure, thereby generating peptide-specific immune effector cells; (b) purifying immune effector cells specific to the peptide, and (c) isolating a TCR sequence from the purified immune effector cells. Also described is a method of making a cell comprising transferring a nucleic acid or expression vector of the disclosure into the cell. Provided herein is an in vitro method for making a therapeutic T cell vaccine comprising co-culturing T cells with a peptide of the disclosure.

Nucleic acids of the disclosure include those that encode for CDR regions, variable regions, engineered TCRs, polypeptides, TCR-a polypeptides, TCR-b polypeptides, peptides, polypeptides, and fusion proteins described herein. The nucleic acid may be RNA. The nucleic acid may also be DNA or a cDNA encoding the peptide or polypeptide, or a complement of the peptide or polypeptide. The nucleic acid may comprise one of SEQ ID NOS: 1, 2, 28, 29, or a fragment thereof. The nucleic acid may comprise a nucleotide having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS: 1, 2, 28, 29, or a fragment thereof. Also provided are compositions comprising the polypeptides, cells, nucleic acids, or engineered TCRs of the disclosure. Also described is a method of making an engineered cell comprising transferring a nucleic acid or vector of the disclosure into a cell. The disclosure also provides for a method for treating cancer in a subject comprising administering a polypeptide, composition, cell, nucleic acid, or engineered TCR to a subject in need thereof. Methods also include methods of reducing tumor burden; methods of lysing a cancer cell; methods of killing tumor/cancerous cells; methods of increasing overall survival; methods of reducing the risk of getting cancer or of getting a tumor; methods of increasing recurrent free survival; methods of preventing cancer; and/or methods of reducing, eliminating, or decreasing the spread or metastasis of cancer, the method comprising administering a polypeptide, composition, cell, nucleic acid, or engineered TCR to a subject in need thereof.

The polypeptide of the disclosure or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 8. The polypeptide may comprise a CDR3 comprising an amino acid sequence of SEQ ID NO: 8. The polypeptide of the disclosure or the TCR-b polypeptide may comprise a CDR3 comprising an amino acid sequence with at least or exactly 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 14. The polypeptide of the disclosure or the TCR-b polypeptide may comprise a CDR3 comprising an amino acid sequence of SEQ ID NO: 14. The engineered TCR may comprise a TCR-a polypeptide comprising a CDR 3 having an amino acid of SEQ ID NO: 8 and a TCR-b polypeptide comprising a CDR3 comprising an amino acid sequence of SEQ ID NO: 14.

The polypeptide of the disclosure or the TCR-a polypeptide may comprise a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 35. The polypeptide may comprise a CDR3 comprising an amino acid sequence of SEQ ID NO: 35. The polypeptide of the disclosure or the TCR-b polypeptide may comprise a CDR3 comprising an amino acid sequence with at least or exactly 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 41. The polypeptide of the disclosure or the TCR-b polypeptide may comprise a CDR3 comprising an amino acid sequence of SEQ ID NO: 41. The engineered TCR may comprise a TCR-a polypeptide comprising a CDR 3 having an amino acid of SEQ ID NO: 35 and a TCR-b polypeptide comprising a CDR3 comprising an amino acid sequence of SEQ ID NO: 41.

The polypeptide may comprise a variable region comprising a CDR1, CDR2, and CDR3 from a TCR-a polypeptide and/or a TCR-b polypeptide. The variable region may comprise a CDR1 with at least 80% sequence identity to SEQ ID NO: 6. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 6. The variable region may comprise a CDR2 with at least 80% sequence identity to SEQ ID NO: 7. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 7. The variable region may comprise a CDR1 comprising the amino acid sequence of SEQ ID NO: 6 and/or a CDR2 comprising the amino acid sequence of SEQ ID NO: 7. The variable region may comprise an amino acid sequence with at least 70% sequence identity to SEQ ID NO: 4. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 4. The variable region may comprise the amino acid sequence of SEQ ID NO: 4. The polypeptide may comprise a T cell receptor alpha (TCR-a) variable region. The polypeptide may comprise a TCR-a variable and constant region. The polypeptide may further comprise a signal peptide. The signal peptide may comprise an amino acid sequence with at least 80% identity to SEQ ID NO: 5. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 5. The signal peptide may comprise an amino acid sequence of SEQ ID NO: 5. The variable region may comprise a CDR1, CDR2, and/or CDR3.

The polypeptide may comprise a variable region comprising a CDR1, CDR2, and CDR3 from a TCR-a polypeptide and/or a TCR-b polypeptide. The variable region may comprise a CDR1 with at least 80% sequence identity to SEQ ID NO: 33. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 33. The variable region may comprise a CDR2 with at least 80% sequence identity to SEQ ID NO: 34. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 34. The variable region may comprise a CDR1 comprising the amino acid sequence of SEQ ID NO: 33 and/or a CDR2 comprising the amino acid sequence of SEQ ID NO: 34. The variable region may comprise an amino acid sequence with at least 70% sequence identity to SEQ ID NO: 31. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 31. The variable region may comprise the amino acid sequence of SEQ ID NO: 31. The polypeptide may comprise a T cell receptor alpha (TCR-a) variable region. The polypeptide may comprise a TCR-a variable and constant region. The polypeptide may further comprise a signal peptide. The signal peptide may comprise an amino acid sequence with at least 80% identity to SEQ ID NO: 32. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 32. The signal peptide may comprise an amino acid sequence of SEQ ID NO: 32. The variable region may comprise a CDR1, CDR2, and/or CDR3.

The variable region may comprise a CDR1 with at least 80% sequence identity to SEQ ID NO: 12. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 12. The variable region may comprise a CDR2 with at least 80% sequence identity to SEQ ID NO: 13. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 13 The variable region may comprise a CDR1 comprising the amino acid sequence of SEQ ID NO: 12 and/or a CDR2 comprising the amino acid sequence of SEQ ID NO: 13. The variable region may comprise an amino acid sequence with at least 70% sequence identity to SEQ ID NO: 10. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 10. The variable region may comprise the amino acid sequence of SEQ ID NO: 10. The polypeptide may comprise a T cell receptor beta (TCR-b) variable region. The polypeptide may comprise a TCR-b variable and constant region. The polypeptide may comprise or further comprise a signal peptide. The signal peptide may comprise an amino acid sequence having at least 80% identity to SEQ ID NO: 11. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 11. The signal peptide comprises the amino acid sequence of SEQ ID NO: 11. The variable region may comprise a CDR1, CDR2, and/or CDR3.

The variable region may comprise a CDR1 with at least 80% sequence identity to SEQ ID NO: 39. The variable region may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 39. The variable region may comprise a CDR2 with at least 80% sequence identity to SEQ ID NO: 40. The variable region may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 40 The variable region may comprise a CDR1 comprising the amino acid sequence of SEQ ID NO: 39 and/or a CDR2 comprising the amino acid sequence of SEQ ID NO: 40. The variable region may comprise an amino acid sequence with at least 70% sequence identity to SEQ ID NO: 37. The variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 37. The variable region may comprise the amino acid sequence of SEQ ID NO: 37. The polypeptide may comprise a T cell receptor beta (TCR-b) variable region. The polypeptide may comprise a TCR-b variable and constant region. The polypeptide may comprise or further comprise a signal peptide. The signal peptide may comprise an amino acid sequence having at least 80% identity to SEQ ID NO: 38. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 38. The signal peptide comprises the amino acid sequence of SEQ ID NO: 38. The variable region may comprise a CDR1, CDR2, and/or CDR3.

The TCR may comprise a TCR-a polypeptide comprising a variable region comprising CDR1, CDR2, and CDR3 and a TCR-b polypeptide comprising a variable region comprising CDR1, CDR2, and CDR3. The TCR-a polypeptide may comprise a CDR1 with at least 80% sequence identity to SEQ ID NO: 6 and/or the TCR-b polypeptide may comprise a CDR1 with at least 80% sequence identity to SEQ ID NO: 12. The TCR-a polypeptide may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 6 and/or the TCR-b polypeptide may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 12. The TCR-a polypeptide may comprise a CDR1 comprising the amino acid sequence of SEQ ID NO: 6 and the TCR-b polypeptide may comprise a CDR1 comprising the amino acid sequence of SEQ ID NO: 12. The TCR-a polypeptide may comprise a CDR2 with at least 80% sequence identity to SEQ ID NO: 7 and the TCR-b polypeptide comprises a CDR2 with at least 80% sequence identity to SEQ ID NO: 13. The TCR-a polypeptide may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 7 and the TCR-b polypeptide comprises a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 13. The TCR-a polypeptide may comprise a CDR2 comprising the amino acid sequence of SEQ ID NO: 7 and the TCR-b polypeptide comprises a CDR2 comprising the amino acid sequence of SEQ ID NO: 13. The CDR1, CDR2, and CDR3 of the TCR-a polypeptide may comprise the amino acid sequence of SEQ ID NO: 6, 7, and 8, respectively and wherein the CDR1, CDR3, and CDR3 of the TCR-b polypeptide may comprise the amino acid sequence of SEQ ID NO: 12, 13, and 14, respectively. The TCR-a variable region may comprise an amino acid sequence with at least 70% sequence identity to SEQ ID NO: 4 and the TCR-b variable region comprises an amino acid sequence with at least 70% sequence identity to SEQ ID NO: 10. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 4 and the TCR-b variable region comprises an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 10. The TCR-a polypeptide may comprise an amino acid sequence having at least 70% sequence identity to SEQ ID NO: 3 and the TCR-b polypeptide comprises an amino acid sequence having at least 70% sequence identity to SEQ ID NO: 9. The TCR-a polypeptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 3 and the TCR-b polypeptide comprises an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 9. The TCR-a and/or TCR-b polypeptide may comprise a signal peptide. The signal peptide may comprise SEQ ID NO: 5 or 11. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 5 or 11.

The TCR may comprise a TCR-a polypeptide comprising a variable region comprising CDR1, CDR2, and CDR3 and a TCR-b polypeptide comprising a variable region comprising CDR1, CDR2, and CDR3. The TCR-a polypeptide may comprise a CDR1 with at least 80% sequence identity to SEQ ID NO: 33 and/or the TCR-b polypeptide may comprise a CDR1 with at least 80% sequence identity to SEQ ID NO: 39. The TCR-a polypeptide may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 33 and/or the TCR-b polypeptide may comprise a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 39. The TCR-a polypeptide may comprise a CDR1 comprising the amino acid sequence of SEQ ID NO: 33 and the TCR-b polypeptide may comprise a CDR1 comprising the amino acid sequence of SEQ ID NO: 39. The TCR-a polypeptide may comprise a CDR2 with at least 80% sequence identity to SEQ ID NO: 34 and the TCR-b polypeptide comprises a CDR2 with at least 80% sequence identity to SEQ ID NO: 40. The TCR-a polypeptide may comprise a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 34 and the TCR-b polypeptide comprises a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 40. The TCR-a polypeptide may comprise a CDR2 comprising the amino acid sequence of SEQ ID NO: 34 and the TCR-b polypeptide comprises a CDR2 comprising the amino acid sequence of SEQ ID NO: 40. The CDR1, CDR2, and CDR3 of the TCR-a polypeptide may comprise the amino acid sequence of SEQ ID NO: 33, 34, and 35, respectively and wherein the CDR1, CDR3, and CDR3 of the TCR-b polypeptide may comprise the amino acid sequence of SEQ ID NO: 39, 40, and 41, respectively. The TCR-a variable region may comprise an amino acid sequence with at least 70% sequence identity to SEQ ID NO: 31 and the TCR-b variable region comprises an amino acid sequence with at least 70% sequence identity to SEQ ID NO: 37. The TCR-a variable region may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 31 and the TCR-b variable region comprises an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 37. The TCR-a polypeptide may comprise an amino acid sequence having at least 70% sequence identity to SEQ ID NO: 30 and the TCR-b polypeptide comprises an amino acid sequence having at least 70% sequence identity to SEQ ID NO: 36. The TCR-a polypeptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 30 and the TCR-b polypeptide comprises an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 36. The TCR-a and/or TCR-b polypeptide may comprise a signal peptide. The signal peptide may comprise SEQ ID NO: 32 or 38. The signal peptide may comprise an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 32 or 38.

The TCR may comprise a modification or is chimeric. The variable region of the TCR may be fused to a TCR constant region that is different from the constant region of the cloned TCR that specifically binds to a peptide of the disclosure. The TCR may exclude a modification described herein.

The TCR-a polypeptide and TCR-b polypeptide may be operably linked. The term “operably linked” can refer to a covalent linkage, such as a peptide bond (e.g. the two elements are polypeptides and are on the same polypeptide), or a non-covalent linkage, such as Van der Waals force (e.g. two polypeptides that have a certain degree of specific binding affinity for each other). The TCR-a polypeptide and TCR-b polypeptide are operably linked through a peptide bond. The TCR-a polypeptide and TCR-b polypeptide are on the same polypeptide and wherein the TCR-b is amino-proximal to the TCR-a. The polypeptide may be further defined as a single-chain TCR. The TCR-a polypeptide and TCR-b polypeptide may be on the same polypeptide and wherein the TCR-a is amino-proximal to the TCR-b. The TCR may comprise a linker between the TCR-a and TCR-b polypeptide. The linker may comprise glycine and serine residues. The linker may be composed of only glycine and serine residues (a glycine-serine linker). The linker may be a flexible linker. Exemplary flexible linkers include glycine polymers (G)n, glycine-serine polymers (including, for example, (GS)n, (GSGGS)n—SEQ ID NO: 18, (G₄S)n and (GGGS)n—SEQ ID NO: 19, where n is an integer of at least one. n may be at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO: 20), GGSGG (SEQ ID NO: 21), GSGSG (SEQ ID NO: 22), GSGGG (SEQ ID NO: 23), GGGSG (SEQ ID NO: 24), GSSSG (SEQ ID NO: 25), and the like. Further linkers useful in the polypeptides and TCRs of the disclosure are described herein. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise. The polypeptide or TCR may exclude having linkers between regions of the polypeptide and TCR described herein.

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in SEQ ID NOS: 6-8 and 12-14. Alternatively, or in combination, a CDR may also be a fragment of a CDR described herein and may lack at least 1, 2, 3, 4, or 5 amino acids from the C-terminal or N-terminal end of a particular CDR sequence.

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in SEQ ID NOS: 33-35 and 39-41. Alternatively, or in combination, a CDR may also be a fragment of a CDR described herein and may lack at least 1, 2, 3, 4, or 5 amino acids from the C-terminal or N-terminal end of a particular CDR sequence.

The TCR or fusion protein may be conjugated to a detection or therapeutic agent. The agent may comprise a fluorescent molecule, radiative molecule, or toxin. The TCR or fusion protein may be conjugated to an agent described herein.

The disclosure also provides for a nucleic acid encoding a TCR-a polypeptide comprising a CDR3 comprising an amino acid sequence with at least 80% sequence identity to SEQ ID NO: 8 and/or a TCR-b polypeptide comprising a CDR3 comprising an amino acid sequence with at least 80% sequence identity to SEQ ID NO: 14. The nucleic acid may encode a TCR-a polypeptide comprising a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 8 and/or the TCR-b polypeptide comprises a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 14. The nucleic acid may encode for a TCR-a polypeptide comprising a CDR1, CDR2, and CDR3 and/or a TCR-b polypeptide comprising a CDR1, CDR2, and CDR3. The nucleic acid may encode for a TCR-a comprising a CDR1 with at least 80% sequence identity to SEQ ID NO: 6 and/or a TCR-b comprising a CDR1 with at least 80% sequence identity to SEQ ID NO: 12. The nucleic acid may encode for a TCR-a comprising a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 6 and/or a TCR-b comprising a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 12. The nucleic acid may encode for a TCR-a comprising a CDR2 with at least 80% sequence identity to SEQ ID NO: 7 and/or a TCR-b comprising a CDR2 with at least 80% sequence identity to SEQ ID NO: 13. The nucleic acid may encode for a TCR-a comprising a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 7 and/or a TCR-b comprising a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 13. The nucleic acid may encode a TCR-a variable region comprising an amino acid sequence with at least 70% sequence identity to SEQ ID NO: 4 and/or a TCR-b variable region comprising an amino acid sequence with at least 70% sequence identity to SEQ ID NO: 10. The nucleic acid may encode a TCR-a variable region comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 4 and/or a TCR-b variable region comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 10. The nucleic acid may encode a TCR-a polypeptide chain comprising the amino acid sequence of SEQ ID NO: 4 and/or a TCR-b chain comprising the amino acid of SEQ ID NO: 10. The nucleic acid may comprise SEQ ID NO: 1 and/or SEQ ID NO: 2. The nucleic acid may comprise a nucleic acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 1 and/or having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 2.

The disclosure also provides for a nucleic acid encoding a TCR-a polypeptide comprising a CDR3 comprising an amino acid sequence with at least 80% sequence identity to SEQ ID NO: 35 and/or a TCR-b polypeptide comprising a CDR3 comprising an amino acid sequence with at least 80% sequence identity to SEQ ID NO: 41. The nucleic acid may encode a TCR-a polypeptide comprising a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 35 and/or the TCR-b polypeptide comprises a CDR3 comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 41. The nucleic acid may encode for a TCR-a polypeptide comprising a CDR1, CDR2, and CDR3 and/or a TCR-b polypeptide comprising a CDR1, CDR2, and CDR3. The nucleic acid may encode for a TCR-a comprising a CDR1 with at least 80% sequence identity to SEQ ID NO: 33 and/or a TCR-b comprising a CDR1 with at least 80% sequence identity to SEQ ID NO: 39. The nucleic acid may encode for a TCR-a comprising a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 33 and/or a TCR-b comprising a CDR1 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 39. The nucleic acid may encode for a TCR-a comprising a CDR2 with at least 80% sequence identity to SEQ ID NO: 34 and/or a TCR-b comprising a CDR2 with at least 80% sequence identity to SEQ ID NO: 40. The nucleic acid may encode for a TCR-a comprising a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 34 and/or a TCR-b comprising a CDR2 having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 40. The nucleic acid may encode a TCR-a variable region comprising an amino acid sequence with at least 70% sequence identity to SEQ ID NO: 31 and/or a TCR-b variable region comprising an amino acid sequence with at least 70% sequence identity to SEQ ID NO: 37. The nucleic acid may encode a TCR-a variable region comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 31 and/or a TCR-b variable region comprising an amino acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 37. The nucleic acid may encode a TCR-a polypeptide chain comprising the amino acid sequence of SEQ ID NO: 31 and/or a TCR-b chain comprising the amino acid of SEQ ID NO: 37. The nucleic acid may comprise SEQ ID NO: 28 and/or SEQ ID NO: 29. The nucleic acid may comprise a nucleic acid sequence having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 28 and/or having or having at least 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 29.

The nucleic acid may comprise a TCR-a (TRA) and TCR-b (TRB) gene. The nucleic acid may be polycistronic. The nucleic acid may also comprise an internal ribosome entry site (IRES) or a P2A linker. The nucleic acid may exclude an IRES or P2A linker. The nucleic acid may comprise a cDNA encoding the TCR-a and/or TCR-b genes. The nucleic acid may encode or further encode for a polypeptide comprising a CD3 binding region. The CD3 binding region may comprise a CD3-specific fragment antigen binding (Fab), single chain variable fragment (scFv), single domain antibody, or single chain antibody.

The peptide may comprise at least 6 contiguous amino acids of a peptide of SEQ ID NO: 15. The peptide may comprise, consist of, or comprise at least 4, 5, 6, 7, 8, or 9 contiguous amino acids of a peptide of SEQ ID NOS: 15. The peptide may comprise or consist of the amino acid sequence of SEQ ID NO: 15. The peptide may be 13 amino acids in length or shorter. The peptide may have at least, at most, exactly, or consist of 6, 7, 8, 9, 10, 11, 12, 13, 14, or 15 amino acids (or any range derivable therein). The peptide may consist of 9 amino acids. The peptide may consist of 8 amino acids. The peptide may consist of 7 amino acids. The peptide may consist of 6 amino acids. The peptide may be immunogenic. The term “immunogenic” may refer to the production of an immune response, such as a protective immune response. The peptide may be modified. The modification may comprise conjugation to a molecule. Modification, such as conjugation to another molecule, may be excluded in the methods, polypeptides, and peptides of the disclosure. The molecule may be an antibody, a lipid, an adjuvant, or a detection moiety (tag). Conjugation to an antibody, a lipid, an adjuvant, or a detection moiety (tag) may be excluded in the polypeptides and peptides of the disclosure. The peptide may comprise 100% sequence identity to a peptide of SEQ ID NO: 15. Peptides of the disclosure also include those that have or have at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to a peptide of SEQ ID NO: 15. The peptide may have or have at least 77% sequence identity to a peptide of SEQ ID NO: 15. The peptide may have or have at least 88% sequence identity to a peptide of SEQ ID NO: 15. The peptides of the disclosure may have 1, 2, or 3 substitutions relative to a peptide of SEQ ID NO: 15. The peptide may have at least or have at most 1, 2, 3, 4, or 5 substitutions relative to a peptide of SEQ ID NO: 15.

The composition may be formulated for parenteral administration, intravenous injection, intramuscular injection, inhalation, or subcutaneous injection. The peptide may be comprised in a liposome, lipid-containing nanoparticle, or in a lipid-based carrier. The compositions of the disclosure may be formulated as a vaccine. The composition may further comprise an adjuvant.

The dendritic cell may comprise a mature dendritic cell. The cell may be a cell with an HLA-A type. The HLA may be a HLA-A, HLA-B, or HLA-C. Cells having a HLA-A, The cell may be an HLA-A0201 type. The cell may be an HLA-A01, HLA-A02, HLA-A11, HLA-A24, HLA-B07, HLA-B08, HLA-B15, or HLA-B40. Cells having a HLA-A, HLA-B, HLA-C, HLA-A0201, HLA-A01, HLA-A02, HLA-A11, HLA-A24, HLA-B07, HLA-B08, HLA-B15, or HLA-B40 may be excluded as cell embodiments of the disclosure.

The method may further comprise isolating the expressed peptide or polypeptide. The T cell may comprise a CD8+ T cell. The T cell may be a CD4+ T cell, a Th1, Th2, Th17, Th9, or Tfh T cell, a cytotoxic T cell, a memory T cell, a central memory T cell, or an effector memory T cell. CD8+ T cells, CD4+ T cells, Th1 T cells, Th2 T cells, Th17 T cells, Th9 T cells, Tfh T cells, cytotoxic T cells, memory T cells, central memory T cells, or effector memory T cells may be excluded from the cell aspects of the disclosure.

The vector may comprise both of the TCR-a and TCR-b genes. The vector may comprise a promoter that directs the expression of the nucleic acid. The promoter may comprise a murine stem cell virus (MSCV) promoter.

The cell may comprise a stem cell, a progenitor cell, an immune cell, or a natural killer (NK) cell. The cell may comprise a hematopoietic stem or progenitor cell, a T cell, a cell differentiated from mesenchymal stem cells (MSCs) or an induced pluripotent stem cell (iPSC). The cell may be isolated or derived from peripheral blood mononuclear cell (PBMCs). The T cell may comprise a cytotoxic T lymphocyte (CTL), a CD8+ T cell, a CD4+ T cell, an invariant NK T (INKT) cell, a gamma-delta T cell, a NKT cell, or a regulatory T cell. The cell may be isolated from a cancer patient. The cell may be isolated from a non-cancerous patient. The cell may be isolated from a healthy patient. The cell may be frozen or may have never been frozen. The cell may be in cell culture. The cell may be one that lacks endogenous expression of TCR genes. The cell may further comprise a chimeric antigen receptor (CAR). A stem cell, a progenitor cell, an immune cell, a natural killer (NK) cell, a hematopoietic stem or progenitor cell, a T cell, a cell differentiated from mesenchymal stem cells (MSCs), an induced pluripotent stem cell (iPSC), a cell isolated or derived from peripheral blood mononuclear cell (PBMCs), a cytotoxic T lymphocyte (CTL), a CD8+ T cell, a CD4+ T cell, an invariant NK T (INKT) cell, a gamma-delta T cell, a NKT cell, a regulatory T cell, a cell isolated from a cancer patient, a cell isolated from a non-cancerous patient, a cell isolated from a healthy patient, a frozen cell, a fresh cell, a cell in culture, a cell that lacks endogenous expression of TCR genes, or a cell that further comprises a chimeric antigen receptor (CAR) may be excluded from the cell aspects of the disclosure.

The composition may have been determined to be serum-free, mycoplasma-free, endotoxin-free, and/or sterile. The method may comprise or further comprise culturing the cell in media, incubating the cell at conditions that allow for the division of the cell, screening the cell, and/or freezing the cell.

The subject may be one that has been diagnosed with cancer, such as a cancer described herein. The cancer may comprise a solid tumor. The cancer may exclude a cancer described herein. The cancer may exclude a solid tumor. The subject may be one that has been previously been treated for the cancer. The subject may be one that has been determined to be resistant to the previous treatment. The method may comprise or further comprise the administration of an additional therapy. The cancer may be further defined as a solid tumor. The cancer may also be a blood cancer, such as leukemia. The cancer may be a cancer described herein. The cancer may be a stage I, II, III, or IV cancer. The cancer may comprise metastatic and/or recurrent cancer. The cancer may exclude a stage I, II, III, or IV cancer, or the cancer may exclude metastatic and/or recurrent cancer. The cancer may comprise a TBXT antigen+ cancer. The cancer may comprise a cancer that expresses a peptide of SEQ ID NO: 15. The subject or patient may be one that has been determined to have TBXT antigen+ cancer cells or cancel cells that are positive for the epitope of SEQ ID NO: 15. The cancer may be a TBXT antigen overexpressing cancer, such as overexpression compared to a non-cancerous subject. The subject or patient may be or may exclude one that has been determined to have TBXT antigen overexpression in a biological sample. The TBXT antigen may comprise or consist of SEQ ID NO: 15. The biological sample may comprise or exclude cancer cells or a biopsy. The subject may be a mammal. The subject may comprise a laboratory test animal, such as a mouse, rat, rabbit, dog, cat, horse, or pig. The subject may be a human. The subject may be one that has been determined to be HLA-A02 and/or HLA-A0201 positive.

The compositions of the disclosure may be formulated as a vaccine. The compositions and methods of the disclosure provide for prophylactic therapies to prevent cancer. The compositions and methods of the disclosure provide for therapeutic therapies to treat existing cancers, such as for the treatment of patients with cancer. The composition may comprise or exclude an adjuvant. Adjuvants are known in the art and include, for example, TLR agonists and aluminum salts.

The methods of the disclosure may comprise or exclude screening the cell for one or more cellular properties, such as for TCR expression, incorporation of nucleic acids encoding TCR genes, or for immunogenic properties, such as binding of the TCR to a cancer antigen such as TBXT antigen or a peptide of SEQ ID NO: 15.

The method may comprise or exclude administering a cell or a composition comprising a cell and wherein the cell comprises an autologous cell. The cell may comprise or exclude a non-autologous cell. The cell may also be allogenic or xenogenic.

Compositions may comprise a MHC polypeptide and a peptide of the disclosure and wherein the MHC polypeptide and/or peptide is conjugated to a detection tag. As such, suitable detection tags include, but are not limited to radioisotopes, fluorochromes, chemiluminescent compounds, dyes, and proteins, including enzymes. The tag may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent tags that produce signals include, but are not limited to bioluminescence and chemiluminescence. Examples of suitable fluorescent tags include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th). Detection tags also include streptavidin or it's binding partner, biotin.

The MHC polypeptide and peptide may be operatively linked. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. Accordingly, the methods, compositions, cells, polypeptides and nucleic acids include wherein the MHC polypeptide and peptide are operatively linked through a peptide bond. Also included is wherein the MHC polypeptide and peptide are operatively linked through van der Waals forces. The peptide-MHC may be operatively linked to form a pMHC complex. At least two pMHC complexes may be operatively linked together. The compositions and methods may include at least, or include at most 2, 3, 4, 5, 6, 7, 8, 9, or 10 pMHC complexes operatively linked to each other. At least two MHC polypeptides may be linked to one peptide. The average ratio of MHC polypeptides to peptides may be 1:1 to 4:1. The ratio or average ratio may be at least, at most, or about 1, 2, 3, 4, 5, or 6 to about 1, 2, 3, 4, 5, or 6 (or any derivable range therein).

The peptide may be complexed with MHC. The MHC may comprise HLA-A, HLA-B, or HLA-C type. The peptides may be loaded onto dendritic cells, lymphoblastoid cells, peripheral blood mononuclear cells (PBMCs), artificial antigen presentation cells (aAPC) or artificial antigen presenting surfaces. The artificial antigen presenting surface may comprise a MHC polypeptide conjugated or linked to a surface. Exemplary surfaces include a bead, microplate, glass slide, or cell culture plate.

Method of the disclosure may comprise or exclude counting the number of T cells bound with peptide and/or MHC. The composition comprising T cells may be isolated from a subject. The subject may be one, as defined herein, such as a human subject. The method may comprise or exclude sorting the number of T cells bound with peptide and/or MHC. Methods of the disclosure may also comprise or exclude sequencing one or more TCR genes from T cells bound with peptide and/or MHC. The method may comprise or exclude sequencing the TCR alpha and/or beta gene(s) from a TCR, such as a TCR that binds to a peptide of the disclosure. Methods may also comprise or exclude grouping of lymphocyte interactions by paratope hotspots (GLIPH) analysis. This is further described in Glanville et al., Nature. 2017 Jul. 6; 547 (7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. The methods may also further comprise isolating the expressed peptide or polypeptide from a cell of the disclosure.

Methods of the disclosure may comprise or further comprise or exclude screening the dendritic cell for one or more cellular properties. The method may further comprise or exclude contacting the cell with one or more cytokines or growth factors. The one or more cytokines or growth factors may comprise GM-CSF. The cellular property may comprise cell surface expression of one or more of CD86, HLA, and CD14. The dendritic cell may be derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. The APCs may be dendritic cells. The dendritic cell may be derived from a peripheral blood monocyte (PBMC). The dendritic cells may be isolated from PBMCs. The dendritic cells or the cells in which the DCs are derived from are isolated by leukaphereses.

Peptide-MHC (pMHC) complexes may be made by contacting a peptide of the disclosure with a MHC complex. The peptide may be expressed in the cell and binds to endogenous MHC complex to form a pMHC. Peptide exchange may be used to make the pMHC complex. For example, cleavable peptides, such as photocleavable peptides may be designed that bind to and stabilize the MHC. Cleavage of the peptide (eg. by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. Also described are pMHC complexes comprising a peptide of the disclosure. The pMHC complex may be operatively linked to a solid support or may be attached to a detectable moiety, such as a fluorescent molecule, a radioisotope, or an antibody. Also described in the disclosure are peptide-MHC multimeric complexes that include, include at least or include at most 1, 2, 3, 4, 5, or 6 peptide-MHC molecules operatively linked together. The linkage may be covalent, such as through a peptide bond, or non-covalent. pMHC molecules may be bound to a biotin molecule. Such pMHC molecules may be multimerized through binding to a streptavidin molecule. pMHC multimers may be used to detect antigen-specific T cells or TCR molecules that are in a composition or in a tissue. The multimers may be used to detect peptide- or peptide-specific T cells in situ or in a biopsy sample. Multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose cancer in subjects or to determine immune responses in individuals with cancer.

The methods may comprise or exclude isolating the starting population of immune effector cells from peripheral blood mononuclear cells (PBMCs). The starting population of immune effector cells may be obtained from a subject. The methods of the disclosure may comprise or exclude introducing the peptides or a nucleic acid encoding the peptide into the dendritic cells prior to the co-culturing. The introduction of the peptide may be done by transfecting or infecting dendritic cells with a nucleic acid encoding the peptide or by incubating the peptide with the dendritic cells. The peptide or nucleic acids encoding the peptide may be introduced by electroporation. Other methods of transfer of nucleic acids are known in the art, such as lipofection, calcium phosphate transfection, transfection with DEAE-dextran, microinjection, and virus-mediated transduction, and are useful in methods of the disclosure for transferring nucleic acids of the disclosure into cells. The peptide or nucleic acids encoding the peptide may be introduced by adding the peptide or nucleic acid encoding the peptide to the dendritic cell culture media. The immune effector cells may be co-cultured with a second population of dendritic cells into which the peptide or the nucleic acid encoding the peptide has been introduced. A population of CD4-positive or CD8-positive and peptide MHC tetramer-positive T cells may be purified from the immune effector cells following the co-culturing. The population of CD4-positive or CD8-positive and peptide MHC tetramer-positive T cells may be purified by fluorescence activated cell sorting (FACS). A clonal population of peptide-specific immune effector cells may be generated by limiting or serial dilution followed by expansion of individual clones by a rapid expansion protocol.

Purifying may further comprise or exclude the generation of a clonal population of peptide-specific immune effector cells by limiting or serial dilution of sorted cells followed by expansion of individual clones by a rapid expansion protocol. Methods of the disclosure may comprise or exclude cloning of a T cell receptor (TCR) from the clonal population of peptide-specific immune effector cells. The term isolating in the methods of the disclosure may be defined or further defined as cloning of a T cell receptor (TCR) from the clonal population of peptide-specific immune effector cells. Cloning of the TCR may be cloning of a TCR alpha and a beta chain. The TCR may be cloned using a 5′-Rapid amplification of cDNA ends (RACE) method. The TCR alpha and beta chains may be cloned using a 5′-Rapid amplification of cDNA ends (RACE) method. The cloned TCR may be subcloned into an expression vector. The expression vector may comprise a linker domain between the TCR alpha sequence and TCR beta sequence. The expression vector may be or may exclude a retroviral or lentiviral vector. The vector may also be or may also exclude an expression vector described herein. The linker domain may comprise or exclude a sequence encoding one or more peptide cleavage sites. The one or more cleavage sites may be a Furin cleavage site and/or a P2A cleavage site. The TCR alpha sequence and TCR beta sequence may be linked by an IRES sequence.

A host cell of the disclosure may be transduced with an expression vector to generate an engineered cell that expresses the TCR alpha and/or beta chains. The host cell may be an immune cell. The immune cell may be a T cell and the engineered cell may be referred to as an engineered T cell. The T cell may be type of T cell described herein, such as a CD8⁺ T cell, CD4+ T cell, or γδ T cell. The starting population of immune effector cells may be obtained from a subject having cancer and the host cell is allogeneic or autologous to the subject. The peptide-specific T cells may be autologous or allogeneic. A population of CD4-positive or CD8-positive and peptide MHC tetramer-positive engineered T cells may be purified from the transduced host cells. A clonal population of peptide-specific engineered T cells may be generated by limiting or serial dilution followed by expansion of individual clones by a rapid expansion protocol. Purifying in the methods of the disclosure may be defined as purifying a population of CD4-positive or CD8-positive and peptide MHC tetramer-positive T cells from the immune effector cells following the co-culturing.

The cancer may be chordoma. The cancer may be small cell lung cancer. Also included is a cancer described herein though any cancer recited herein may be excluded.

The peptide may be linked to a solid support. The peptide may be conjugated to the solid support or is bound to an antibody that is conjugated to the solid support. The solid support may comprise a microplate, a bead, a glass surface, a slide, or a cell culture dish. The solid support may comprise a nanofluidic chip. Detecting T cell responses may comprise or further comprise detecting the binding of the peptide to the T cell or TCR. Detecting T cell responses may comprise an ELISA, ELISPOT, or a tetramer assay.

Methods of the disclosure may also be used for determining the efficacy of a vaccine, such as a cancer vaccine.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C. Parallel reaction monitoring mass spectrometry (PRM-MS) identification of TBXT-345 peptide. MHC peptide eluted from (B) K562-A2-TBXT (A0201 and TBXT forced expressing), as well as (A) K562-A2-GFP (A0201 and GFP forced expressing, negative control) with immuno-precipitation (IP) protocol were used for PRM-MS analysis. Pierce™ Peptide Retention Time Calibration Mixture peptides were used to monitor retention time drifts and adjust the scheduled PRM method. (C) TBXT-345 synthesis peptide was spiked into negative sample (K562-A2-GFP) and analyzed with PRM-MS for the alignment. An inclusion list containing m/z values of protonated precursor peptide ions of interest was generated in Skyline-daily. TBXT-345 peptide peak was detected in K562-A2-TBXT sample (B) and comparable with synthesis peptide (C). No peak was detected in negative sample (A).

FIG. 2. TBXT-345 peptide (SQYPSLWSV, HLA-A0201) specific CTL generation.

FIG. 3A-J. Functional validation of TBXT-345 specific CTL cell lines. T2 cells pulsed with various concentrations of TBXT-345 peptide were used as targets. The lysis ability of TBXT-345 CTL cell lines was detected with 4 hour Cr51 release assay (CRA). The effector to target (E:T) ratio is 20:1. (B) Melanoma cell line A375 (TBXT−, HLA-A0201+) was used as a control cell line. (C-F) K562-A2-GFP (A0201+, TBXT−), K562-A2-TBXT (A0201+, TBXT+), H460 (A0201−, TBXT+), H460-A2 (A0201+, TBXT+) were used as the target to test TBXT-345 CTL recognition using 4 hour CRA. (G-J) Chordoma cell lines UM-Chor-1, UM-Chor-5C, U-CHCF360 (A0201+, TBXT+), and MUG-CC1 (A0201−, TBXT+) were used as the targets to test TBXT-345 CTL recognition using 20 hour CRA.

FIG. 4A-B. Cold target inhibition assay to verify the recognizing specificity of TBXT-345 specific CTL. K562-A2-TBXT (A) and H460-A2 (B) labeled with 51Cr were used as hot targets. The T2 cells pulsed with TBXT-345 peptide without 51Cr labeling were used as cold target. T2 cells pulsed with irrelevant peptide M26 were used as control cold target. The E:T is 10:1. The cold target: Hot target is 10:1 or 20:1. The killing inhibition of cold target to TBXT-345 peptide specific CTL was detected with 4 hour CRA.

FIG. 5A-C. MHC blocking assay of TBXT-345 specific CTL to chordoma cells with CRA. Chordoma cell line (A) UM-Chor-1, (B) U-CHCF365 and (C) UM-Chor-5C labeled with 51Cr were co-cultured with TBXT-345 CTL in the presence of anti-HLA-ABC (50 μg/ml) or isotype antibody. The E:T is 20:1. The cytotoxic lysis of TBXT-345 specific CTL to each chordoma cell line was detected with 20 hour CRA.

FIG. 6. MHC blocking assay of TBXT-345 specific CTL to chordoma cells with fluorescence detection.

FIG. 7: TBXT-345 peptide specific TCR-T (clone D12) generation.

FIG. 8A-F. TBXT-345 peptide specific D12 TCR-T functional validation with CRA. (A) T2 cells pulsed with various concentrations of TBXT-345 peptide were used as targets. The lysis ability of TBXT-345 peptide specific D12 TCR-T was detected with 4 hour Cr51 release assay (CRA). The effector to target (E:T) ratio is 20:1. (B) A375 melanoma cell line (A0201+, TBXT−), (C) K562-A2-GFP (A0201+, TBXT−), (D) K562-A2-TBXT (A0201+, TBXT+), (E) H460 (A0201−, TBXT+), and (F) H460-A2 (A0201+, TBXT+) were used as targets to test the recognition of TBXT-345 peptide specific TCR-T with 4 hour CRA. The effector to target (E:T) ratio is from 40:1 to 1.25:1.

FIG. 9A-B. Functional detection of TBXT-345 peptide specific D12 TCR-T with intracellular cytokine staining (ICS) assay. The TBXT-345 peptide specific TCR-T cell line was co-cultured with T2+M26 peptide, T2+TBXT-345 peptide, cancer cell line A375, K562-A2-GFP, K562-A2-TBXT, H460, H460-A2 with E:T=10:1 ratio. After overnight co-culturing, the TCR pathway down-stream activated marker, CD137, CD69, IFN-γ and TNF-α were detected with ICS assay.

FIG. 10A-D. Killing assay of TBXT-345 peptide specific D12 TCR-T to chordoma cell lines. Chordoma cell line (A) UM-Chor-1, (B) UM-Chor-5C, (C) U-CHCF365 (all are A0201+, TBXT+), and (D) MUC-CC1 (A0201−, TBXT+), were used as targets to test the recognition of TBXT-345 peptide specific TCR-T with 20 hour CRA. The effector to target (E:T) ratio is from 40:1 to 1.25:1.

FIG. 11A-C. MHC blocking assay of TBXT-345 specific D12 TCR-T to chordoma cells with CRA. Chordoma cell line (A) UM-Chor-1, (B) U-CHCF365 and (C) UM-Chor-5C labeled with 51Cr were co-cultured with TBXT-345 specific TCR-T in the presence of anti-HLA-ABC (50 μg/ml) or isotype antibody. The E:T is 20:1. The cytotoxic lysis of TBXT-345 specific TCR-T to each chordoma cell line was detected with 20 hour CRA.

FIG. 12. MHC blocking assay of TBXT-345 D12 TCR-T to chordoma cells with fluorescence detection.

FIG. 13. TBXT-345 B4 TCR-T generation. The whole length of TBXT-345 B4 clone specific TCR DNA fragment was cloned into retroviral vector pMSGV1 and the recombinant retrovirus was generated. The PBMC from HLA-A0201 healthy donor was infected with recombinant retrovirus. After infection, the tetramer+/CD8+ population were sorted and expanded. After expansion, high purity of TBXT-345 specific TCR-T was obtained.

FIG. 14A-E. TBXT-345 B4 TCR-T functional validation with CRA. FIG. 14A shows T2 cells pulsed with various concentrations of TBXT-345 peptide were used as targets. The lysis ability of TBXT-345 B4 TCR-T was detected with 4 hour Cr51 release assay (CRA). The effector to target (E:T) ratio was 20:1. Lung cancer cell lines H460 (FIG. 14B; A0201−, TBXT+), and H460-A2 (FIG. 14C; A0201+, TBXT+), as well as leukemia cell lines K562-A2-GFP (FIG. 14D; A0201+, TBXT−), K562-A2-TBXT (FIG. 14E; A0201+, TBXT+), were used as targets to test the recognition of TBXT-345 B4 TCR-T with 4 hour CRA. The effector to target (E:T) ratio ranged from 40:1 to 1.25:1.

FIG. 15. Functional detection of TBXT-345 B4 TCR-T with intracellular cytokine staining (ICS) assay. The TBXT-345 B4 TCR-T cells was co-cultured with T2+M26 peptide, T2+TBXT-345 peptide, cancer cell line A375, K562-A2-GFP, K562-A2-TBXT, H460, H460-A2 with E:T=10:1 ratio. After overnight co-culturing, the TCR pathway down-stream activated marker, CD137, CD69, IFN-γ and TNF-α were detected with ICS assay.

FIG. 16A-D. Killing assay of TBXT-345 B4 TCR-T to chordoma cell lines. Chordoma cell lines U-CH1 (FIG. 16A), UM-Chor-1 (FIG. 16B), U-CHCF365 (FIG. 16C), UM-Chor-5C (FIG. 16D) (all are A0201+, TBXT+), were as targets to test the recognition of TBXT-345 B4 TCR-T with 20 hour CRA. The effector to target (E:T) ratio ranged from 40:1 to 1.25:1.

FIG. 17. Functional detection of TBXT-345 B4 TCR-T to chordoma cells with intracellular cytokine staining (ICS) assay. The TBXT-345 B4 TCR-T cells were co-cultured with chordoma cell lines U-CH1, U-CHCF365, UM-Chor-1 and UM-Chor-5C with E:T=10:1 ratio. After overnight co-culturing, the TCR pathway down-stream activated marker, CD137, CD69, IFN-γ and TNF-α were detected with ICS assay.

### DETAILED DESCRIPTION OF THE INVENTION

The present disclosure provides a T-cell receptor (TCR) which recognizes a HLA-A0201 restricted peptide from the TBXT (Brachyury) antigen having the amino acid sequence SQYPSLWSV (SEQ ID NO: 15). TBXT (Brachyury) is uniquely expressed in chordoma (about 90% cases), but not any normal tissues. Thus, TBXT is a potential valuable target for chordoma. Described herein is the A0201 restricted peptide of SEQ ID NO: 15, as well as specific functional T cell receptor-engineered T cells (TCR-T) targeting TBXT. The compositions and methods provide for immunotherapeutic treatments for patients having chordoma and other tumors that express TBXT, such as small cell lung cancer (about 50% cases show overexpression of TBXT). Treatments include peptide vaccination as well as TCR-T cell therapy.

### I. ENGINEERED T CELL RECEPTORS

T-cell receptors comprise two different polypeptide chains, termed the T-cell receptor α (TCRα) and β (TCRβ) chains, linked by a disulfide bond. These a: β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Ca domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of a helix. The intramolecular disulfide bond, which in immunoglobulin-like domains normally joins two β strands, in a Ca domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

The disclosure provides for engineered T cell receptors. The term “engineered” refers to T cell receptors that have TCR variable regions grafted onto TCR constant regions to make a chimeric polypeptide that binds to peptides and antigens of the disclosure. The TCR may comprise intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

The TCR may comprise non-TCR sequences. Accordingly, methods, compositions, nucleic acids, polypeptides, and cells of the disclosure relate to TCRs with sequences that are not from a TCR gene. The TCR may be chimeric, in that it contains sequences normally found in a TCR gene, but contains sequences from at least two TCR genes that are not necessarily found together in nature.

In some aspects, the TCR may be the TBXT-345 specific TCR identified as TCR D12, for example as described herein and in the Examples. Sequences and characteristics of TCR D12 are set forth in Table 1, Table 2, and Table 3. In some embodiments, the TCR is encoded by or comprises the sequence of any of SEQ ID NOS: 1-14, 16, 17, 26, and 27, or a sequence having at least 80% identity thereto. In some embodiments, the TCR is encoded by or comprises any of the sequences set forth in Table 1, Table 2, or Table 3, or a sequence having at least 80% identity thereto.

In some aspects, the TCR may be the TBXT-345 specific TCR identified as TCR B4, for example as described herein and in the Examples. Sequences and characteristics of TCR B4 are set forth in Table 4, Table 5, and Table 6. In some embodiments, the TCR is encoded by or comprises the sequence of any of SEQ ID NOS: 28-45, or a sequence having at least 80% identity thereto. In some embodiments, the TCR is encoded by or comprises any of the sequences set forth in Table 4, Table 5, or Table 6, or a sequence having at least 80% identity thereto.

The engineered TCRs of the disclosure may comprise an amino acid or nucleic acid sequence as shown below:

TABLE 1
TBXT-345 TCR D12 Sequences

| | | SEQ ID |
|---|---|---|
| Description | Sequence | NO: |
| Alpha Chain | ATGGCATGCCCTGGCTTCCTGTGGGCACTTGTGATCTCCAC | 1 |
| (TRAV38- | CTGTCTTGAATTTAGCATGGCTCAGACAGTCACTCAGTCTC | |
| 2/DV8*01 F) | AACCAGAGATGTCTGTGCAGGAGGCAGAGACCGTGACCC | |
| DNA | TGAGCTGCACATATGACACCAGTGAGAGTGATTATTATTT | |
| | ATTCTGGTACAAGCAGCCTCCCAGCAGGCAGATGATTCTC | |
| | GTTATTCGCCAAGAAGCTTATAAGCAACAGAATGCAACAG | |
| | AGAATCGTTTCTCTGTGAACTTCCAGAAAGCAGCCAAATC | |
| | CTTCAGTCTCAAGATCTCAGACTCACAGCTGGGGGATGCC | |
| | GCGATGTATTTCTGTGCTCTAGATTCAGGAGGAGGTGCTG | |
| | ACGGACTCACCTTTGGCAAAGGGACTCATCTAATCATCCA | |
| | GCCCTATATCCAGAACCCTGACCCTGCCGTGTACCAGCTG | |
| | AGAGACTCTAAATCCAGTGACAAGTCTGTCTGCCTATTCA | |
| | CCGATTTTGATTCTCAAACAAATGTGTCACAAAGTAAGGA | |
| | TTCTGATGTGTATATCACAGACAAAACTGTGCTAGACATG | |
| | AGGTCTATGGACTTCAAGAGCAACAGTGCTGTGGCCTGGA | |
| | GCAACAAATCTGACTTTGCATGTGCAAACGCCTTCAACAA | |
| | CAGCATTATTCCAGAAGACACCTTCTTCCCCAGCCCAGAA | |
| | AGTTCCTGTGATGTCAAGCTGGTCGAGAAAAGCTTTGAAA | |
| | CAGATACGAACCTAAACTTTCAAAACCTGTCAGTGATTGG | |
| | GTTCCGAATCCTCCTCCTGAAAGTGGCCGGGTTTAATCTGC | |
| | TCATGACGCTGCGGCTGTGGTCCAGCTAA | |
| Beta chain | ATGGGTACCAGTCTCCTATGCTGGGTGGTCCTGGGTTTCCT | 2 |
| (TRBV7-7*01 | AGGGACAGATTCTGTTTCCACAGATCACACAGGTGCTGGA | |
| F) DNA | GTCTCCCAGTCTCCCAGGTACAAAGTCACAAAGAGGGGAC | |
| | AGGATGTAACTCTCAGGTGTGATCCAATTTCGAGTCATGC | |
| | AACCCTTTATTGGTATCAACAGGCCCTGGGGCAGGGCCCA | |
| | GAGTTTCTGACTTACTTCAATTATGAAGCTCAACCAGACA | |
| | AATCAGGGCTGCCCAGTGATCGGTTCTCTGCAGAGAGGCC | |
| | TGAGGGATCCATCTCCACTCTGACGATTCAGCGCACAGAG | |
| | CAGCGGGACTCAGCCATGTATCGCTGTGCCAGCAGCTCGC | |
| | ACCTGGACGGATATAATGAGCAGTTCTTCGGGCCAGGGAC | |
| | ACGGCTCACCGTGCTAGAGGACCTGAAAAACGTGTTCCCA | |
| | CCCGAGGTCGCTGTGTTTGAGCCATCAGAAGCAGAGATCT | |
| | CCCACACCCAAAAGGCCACACTGGTGTGCCTGGCCACAGG | |
| | CTTCTACCCCGACCACGTGGAGCTGAGCTGGTGGGTGAAT | |
| | GGGAAGGAGGTGCACAGTGGGGTCAGCACAGACCCGCAG | |
| | CCCCTCAAGGAGCAGCCCGCCCTCAATGACTCCAGATACT | |
| | GCCTGAGCAGCCGCCTGAGGGTCTCGGCCACCTTCTGGCA | |
| | GAACCCCCGCAACCACTTCCGCTGTCAAGTCCAGTTCTAC | |
| | GGGCTCTCGGAGAATGACGAGTGGACCCAGGATAGGGCC | |
| | AAACCCGTCACCCAGATCGTCAGCGCCGAGGCCTGGGGTA | |
| | GAGCAGACTGTGGCTTTACCTCGGTGTCCTACCAGCAAGG | |
| | GGTCCTGTCTGCCACCATCCTCTATGAGATCCTGCTAGGG | |
| | AAGGCCACCCTGTATGCTGTGCTGGTCAGCGCCCTTGTGTT | |
| | GATGGCCATGGTCAAGAGAAAGGATTTCTAA | |
| Alpha Chain | MACPGFLWALVISTCLEFSMAQTVTQSQPEMSVQEAETVTLS | 3 |
| (TRAV38- | CTYDTSESDYYLFWYKQPPSRQMILVIRQEAYKQQNATENR | |
| 2/DV8*01 F) | FSVNFQKAAKSFSLKISDSQLGDAAMYFCALDSGGGADGLT | |
| Protein | FGKGTHLIIQPYIQNPDPAVYQLRDSKSSDKSVCLFTDFDSQT | |
| | NVSQSKDSDVYITDKTVLDMRSMDFKSNSAVAWSNKSDFA | |
| | CANAFNNSIIPEDTFFPSPESSCDVKLVEKSFETDTNLNFQNLS | |
| | VIGFRILLLKVAGFNLLMTLRLWSS | |
| Alpha - | AQTVTQSQPEMSVQEAETVTLSCTYDTSESDYYLFWYKQPP | 4 |
| variable region | SRQMILVIRQEAYKQQNATENRFSVNFQKAAKSFSLKISDSQ | |
| | LGDAAMYFCALDSGGGADGLTFGKGTHLIIQPY | |
| Alpha - Signal | MACPGFLWALVISTCLEFSM | 5 |
| peptide | | |
| Alpha - CDR1 | TSESDYY | 6 |
| Alpha - CDR2 | QEAYKQQN | 7 |
| Alpha - CDR3 | ALDSGGGADGLT | 8 |
| Beta chain | MGTSLLCWVVLGFLGTDSVSTDHTGAGVSQSPRYKVTKRG | 9 |
| (TRBV7-7*01 | QDVTLRCDPISSHATLYWYQQALGQGPEFLTYFNYEAQPDK | |
| F) Protein | SGLPSDRFSAERPEGSISTLTIQRTEQRDSAMYRCASSSHLDG | |
| | YNEQFFGPGTRLTVLEDLKNVFPPEVAVFEPSEAEISHTQKAT | |
| | LVCLATGFYPDHVELSWWVNGKEVHSGVSTDPQPLKEQPAL | |
| | NDSRYCLSSRLRVSATFWQNPRNHFRCQVQFYGLSENDEWT | |
| | QDRAKPVTQIVSAEAWGRADCGFTSVSYQQGVLSATILYEIL | |
| | LGKATLYAVLVSALVLMAMVKRKDF | |
| Beta - variable | GAGVSQSPRYKVTKRGQDVTLRCDPISSHATLYWYQQALGQ | 10 |
| region | GPEFLTYFNYEAQPDKSGLPSDRFSAERPEGSISTLTIQRTEQR | |
| | DSAMYRCASSSHLDGYNEQFFGPGTRLTVLE | |
| Beta - Signal | MGTSLLCWVVLGFLGTDSVSTDHT | 11 |
| peptide | | |
| Beta - CDR1 | SSHAT | 12 |
| Beta - CDR2 | FNYEAQ | 13 |
| Beta - CDR3 | ASSSHLDGYNEQF | 14 |
| Alpha - | IQNPDPAVYQLRDSKSSDKSVCLFTDFDSQTNVSQSKDSDVY | 16 |
| constant | ITDKTVLDMRSMDFKSNSAVAWSNKSDFACANAFNNSIIPED | |
| region | TFFPSPESSCDVKLVEKSFETDTNLNFQNLSVIGFRILLLKVAG | |
| | FNLLMTLRLWSS | |
| Beta - constant | DLKNVFPPEVAVFEPSEAEISHTQKATLVCLATGFYPDHVELS | 17 |
| region | WWVNGKEVHSGVSTDPQPLKEQPALNDSRYCLSSRLRVSAT | |
| | FWQNPRNHFRCQVQFYGLSENDEWTQDRAKPVTQIVSAEA | |
| | WGRADCGFTSVSYQQGVLSATILYEILLGKATLYAVLVSALV | |
| | LMAMVKRKDF | |

TABLE 2
Features of the D12 TCR-a aspects:

| | Productive TRA rearranged sequence (no stop | | |
|---|---|---|---|
| Result summary: a | codon and in-frame junction) | | |
| V-GENE and allele | Homsap TRAV38- | score = | identity = |
| | 2/DV8*01 F | 1380 | 100.00% (276/276 nt) |
| J-GENE and allele | Homsap TRAJ45*01 F | score = | identity = 96.97% |
| | | 312 | (64/66 nt) |
| FR-IMGT lengths, CDR- | [26.17.34.11] | [7.8.12] | CALDSGGGADGLTF |
| IMGT lengths and AA | | | (SEQ ID NO: 26) |
| JUNCTION | | | |

TABLE 3
Features of the D12 TCR-b aspects:

| | Productive TRB rearranged sequence (no stop codon | | |
|---|---|---|---|
| Result summary: a | and in-frame junction) | | |
| V-GENE and allele | Homsap TRBV7- | score = | identity = |
| | 7*01 F | 1380 | 100.00% (276/276 nt) |
| J-GENE and allele | Homsap TRBJ2- | score = | identity = 92.00% (46/50 |
| | 1*01 F | 214 | nt) |
| D-GENE and allele by | Homsap | D-REGION is in reading frame 3 | |
| IMGT/JunctionAnalysis | TRBD1*01 F | | |
| FR-IMGT lengths, CDR-IMGT | [26.17.38.10] | [5.6.13] | CASSSHLDGYNEQFF |
| lengths and AA JUNCTION | | | (SEQ ID NO: 27) |

TABLE 4
TBXT-345 TCR B4 Sequences

| | | SEQ |
|---|---|---|
| Description | Sequence | ID NO: |
| Alpha Chain | ATGAAGAAGCTACTAGCAATGATTCTGTGGCTTCAACTAG | 28 |
| (TRAV39*01 | ACCGGTTAAGTGGAGAGCTGAAAGTGGAACAAAACCCTC | |
| F) DNA | TGTTCCTGAGCATGCAGGAGGGAAAAAACTATACCATCTA | |
| | CTGCAATTATTCAACCACTTCAGACAGACTGTATTGGTAC | |
| | AGGCAGGATCCTGGGAAAAGTCTGGAATCTCTGTTTGTGT | |
| | TGCTATCAAATGGAGCAGTGAAGCAGGAGGGACGATTAA | |
| | TGGCCTCACTTGATACCAAAGCCCGTCTCAGCACCCTCCA | |
| | CATCACAGCTGCCGTGCATGACCTCTCTGCCACCTACTTCT | |
| | GTGCCGTGGACGACAATGCCAGACTCATGTTTGGAGATGG | |
| | AACTCAGCTGGTGGTGAAGCCCAATATCCAGAACCCTGAC | |
| | CCTGCCGTGTACCAGCTGAGAGACTCTAAATCCAGTGACA | |
| | AGTCTGTCTGCCTATTCACCGATTTTGATTCTCAAACAAAT | |
| | GTGTCACAAAGTAAGGATTCTGATGTGTATATCACAGACA | |
| | AAACTGTGCTAGACATGAGGTCTATGGACTTCAAGAGCAA | |
| | CAGTGCTGTGGCCTGGAGCAACAAATCTGACTTTGCATGT | |
| | GCAAACGCCTTCAACAACAGCATTATTCCAGAAGACACCT | |
| | TCTTCCCCAGCCCAGAAAGTTCCTGTGATGTCAAGCTGGT | |
| | CGAGAAAAGCTTTGAAACAGATACGAACCTAAACTTTCAA | |
| | AACCTGTCAGTGATTGGGTTCCGAATCCTCCTCCTGAAAG | |
| | TGGCCGGGTTTAATCTGCTCATGACGCTGCGGCTGTGGTC | |
| | CAGCTAA | |
| Beta chain | ATGCTGAGTCTTCTGCTCCTTCTCCTGGGACTAGGCTCTGT | 29 |
| (TRBV29-1*01 | GTTCAGTGCTGTCATCTCTCAAAAGCCAAGCAGGGATATC | |
| F) DNA | TGTCAACGTGGAACCTCCCTGACGATCCAGTGTCAAGTCG | |
| | ATAGCCAAGTCACCATGATGTTCTGGTACCGTCAGCAACC | |
| | TGGACAGAGCCTGACACTGATCGCAACTGCAAATCAGGGC | |
| | TCTGAGGCCACATATGAGAGTGGATTTGTCATTGACAAGT | |
| | TTCCCATCAGCCGCCCAAACCTAACATTCTCAACTCTGACT | |
| | GTGAGCAACATGAGCCCTGAAGACAGCAGCATATATCTCT | |
| | GCAGCGTGGAGATTGGGACGGCCAACAATGAGCAGTTCTT | |
| | CGGGCCAGGGACACGGCTCACCGTGCTAGAGGACCTGAA | |
| | AAACGTGTTCCCACCCGAGGTCGCTGTGTTTGAGCCATCA | |
| | GAAGCAGAGATCTCCCACACCCAAAAGGCCACACTGGTGT | |
| | GCCTGGCCACAGGCTTCTACCCCGACCACGTGGAGCTGAG | |
| | CTGGTGGGTGAATGGGAAGGAGGTGCACAGTGGGGTCAG | |
| | CACAGACCCGCAGCCCCTCAAGGAGCAGCCCGCCCTCAAT | |
| | GACTCCAGATACTGCCTGAGCAGCCGCCTGAGGGTCTCGG | |
| | CCACCTTCTGGCAGAACCCCCGCAACCACTTCCGCTGTCA | |
| | AGTCCAGTTCTACGGGCTCTCGGAGAATGACGAGTGGACC | |
| | CAGGATAGGGCCAAACCCGTCACCCAGATCGTCAGCGCCG | |
| | AGGCCTGGGGTAGAGCAGACTGTGGCTTTACCTCGGTGTC | |
| | CTACCAGCAAGGGGTCCTGTCTGCCACCATCCTCTATGAG | |
| | ATCCTGCTAGGGAAGGCCACCCTGTATGCTGTGCTGGTCA | |
| | GCGCCCTTGTGTTGATGGCCATGGTCAAGAGAAAGGATTT | |
| | CTAA | |
| Alpha Chain | MKKLLAMILWLQLDRLSGELKVEQNPLFLSMQEGKNYTIYC | 30 |
| (TRAV39*01 | NYSTTSDRLYWYRQDPGKSLESLFVLLSNGAVKQEGRLMAS | |
| F) Protein | LDTKARLSTLHITAAVHDLSATYFCAVDDNARLMFGDGTQL | |
| | VVKPNIQNPDPAVYQLRDSKSSDKSVCLFTDFDSQTNVSQSK | |
| | DSDVYITDKTVLDMRSMDFKSNSAVAWSNKSDFACANAFN | |
| | NSIIPEDTFFPSPESSCDVKLVEKSFETDTNLNFQNLSVIG | |
| | FRILLLKVAGFNLLMTLRLWSS | |
| Alpha - | ELKVEQNPLFLSMQEGKNYTIYCNYSTTSDRLYWYRQDPGK | 31 |
| variable region | SLESLFVLLSNGAVKQEGRLMASLDTKARLSTLHITAAVHDL | |
| | SATYFCAVDDNARLMFGDGTQLVVKPN | |
| Alpha - Signal | MKKLLAMILWLQLDRLSG | 32 |
| peptide | | |
| Alpha - CDR1 | TTSDR | 33 |
| Alpha - CDR2 | LLSNGAV | 34 |
| Alpha - CDR3 | AVDDNARLM | 35 |
| Beta chain | MLSLLLLLLGLGSVFSAVISQKPSRDICQRGTSLTIQCQVDSQ | 36 |
| (TRBV29-1*01 | VTMMFWYRQQPGQSLTLIATANQGSEATYESGFVIDKFPISR | |
| F) Protein | PNLTFSTLTVSNMSPEDSSIYLCSVEIGTANNEQFFGPGTRLTV | |
| | LEDLKNVFPPEVAVFEPSEAEISHTQKATLVCLATGFYPDHV | |
| | ELSWWVNGKEVHSGVSTDPQPLKEQPALNDSRYCLSSRLRV | |
| | SATFWQNPRNHFRCQVQFYGLSENDEWTQDRAKPVTQIVSA | |
| | EAWGRADCGFTSVSYQQGVLSATILYEILLGKATLYAVLVSA | |
| | LVLMAMVKRKDF | |
| Beta - variable | SAVISQKPSRDICQRGTSLTIQCQVDSQVTMMFWYRQQPGQS | 37 |
| region | LTLIATANQGSEATYESGFVIDKFPISRPNLTFSTLTVSNMSPE | |
| | DSSIYLCSVEIGTANNEQFFGPGTRLTVL | |
| Beta - Signal | MLSLLLLLLGLGSVF | 38 |
| peptide | | |
| Beta - CDR1 | SQVTM | 39 |
| Beta - CDR2 | ANQGSEA | 40 |
| Beta - CDR3 | SVEIGTANNEQF | 41 |
| Alpha - | IQNPDPAVYQLRDSKSSDKSVCLFTDFDSQTNVSQSKDSDVY | 42 |
| constant region | ITDKTVLDMRSMDFKSNSAVAWSNKSDFACANAFNNSIIPED | |
| | TFFPSPESSCDVKLVEKSFETDTNLNFQNLSVIGFRILLLKVAG | |
| | FNLLMTLRLWSS | |
| Beta - constant | EDLKNVFPPEVAVFEPSEAEISHTQKATLVCLATGFYPDHVE | 43 |
| region | LSWWVNGKEVHSGVSTDPQPLKEQPALNDSRYCLSSRLRVS | |
| | ATFWQNPRNHFRCQVQFYGLSENDEWTQDRAKPVTQIVSAE | |
| | AWGRADCGFTSVSYQQGVLSATILYEILLGKATLYAVLVSAL | |
| | VLMAMVKRKDF | |

TABLE 5
Features of the TCR-a aspects:

| | Productive TRA rearranged sequence (no stop | | |
|---|---|---|---|
| Result summary: 1 | codon and in-frame junction) | | |
| V-GENE and allele | Homsap TRAV39*01 F | score = | identity = 100.00% |
| | | 1335 | (267/267 nt) |
| J-GENE and allele | Homsap TRAJ31*01 F | score = | identity = 98.08% |
| | | 251 | (51/52 nt) |
| FR-IMGT lengths, CDR- | [26.17.34.11] | [5.7.9] | CAVDDNARLMF (SEQ |
| IMGT lengths and AA | | | ID NO: 44) |
| JUNCTION | | | |

TABLE 6
Features of the TCR-b aspects:

| | Productive TRB rearranged sequence (no stop codon | | |
|---|---|---|---|
| Result summary: 1 | and in-frame junction) | | |
| V-GENE and allele | Homsap | score = | identity = 100.00% |
| | TRBV29-1*01 F | 1395 | (279/279 nt) |
| J-GENE and allele | Homsap TRBJ2- | score = | identity = 94.00% |
| | 1*01 F | 223 | (47/50 nt) |
| D-GENE and allele by | Homsap | D-REGION is in reading frame 1 | |
| IMGT/JunctionAnalysis | TRBD2*01 F | | |
| FR-IMGT lengths, CDR-IMGT | [26.17.38.10] | [5.7.12] | CSVEIGTANNEQFF |
| lengths and AA JUNCTION | | | (SEQ ID NO: 45) |

### II. PROTEINACEOUS COMPOSITIONS

As used herein, a “protein” “peptide” or “polypeptide” refers to a molecule comprising at least five amino acid residues. As used herein, the term “wild-type” refers to the endogenous version of a molecule that occurs naturally in an organism. Wild-type versions of a protein or polypeptide may be employed, however, a modified protein or polypeptide may be employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. A modified/variant protein or polypeptide may have at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solidphase peptide synthesis (SPPS) or other in vitro methods. The disclosure provides for isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-45. The peptide or polypeptide may be a human sequence or based on a human sequence. The peptide or polypeptide may be one that is not naturally occurring and/or is in a combination of peptides or polypeptides.

The protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-45.

The protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-45 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS: 1-45.

The protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS: 1-45.

The polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS: 1-45 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS: 1-45.

The disclosure provides for a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS: 1-45 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS: 1-45.

The amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of one of SEQ ID NOS: 1-45 may be substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. Areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The greatest local average hydrophilicity of a protein, as governed by the hydrophilicity of its adjacent amino acids, may correlate with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

Amino acid substitutions may be made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions may be made in the naturally occurring sequence. Substitutions can be made in that portion of the antibody that lies outside the domain(s) forming intermolecular contacts. Conservative amino acid substitutions can be used that do not substantially change the structural characteristics of the protein or polypeptide (e.g., one or more replacement amino acids that do not disrupt the secondary structure that characterizes the native antibody).

### III. NUCLEIC ACIDS

Nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides may include regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

The disclosure provides for polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). The isolated polynucleotide may comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5×sodium chloride/sodium citrate (SSC), 0.5% SDS, 1.0 mM EDTA (pH 8.0), hybridization buffer of about 50% formamide, 6×SSC, and a hybridization temperature of 55 °C (or other similar hybridization solutions, such as one containing about 50% formamide, with a hybridization temperature of 42 °C), and washing conditions of 60 °C in 0.5×SSC, 0.1% SDS. A stringent hybridization condition hybridizes in 6×SSC at 45 °C, followed by one or more washes in 0.1×SSC, 0.2% SDS at 68 °C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. One or more particular amino acid residues may be changed using, for example, a site-directed mutagenesis protocol. One or more randomly selected residues may be changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

Nucleic acid molecules may be suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

The nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, e.g., Gaily Kivi et al., BMC Biotechnol. 16:2 (2016). The nucleic acid molecules may be oligonucleotides. The oligonucleotides may be from highly variable regions of the heavy and light or alpha and beta chains of the antibody or TCR of interest. The oligonucleotides may encode all or part of one or more of the CDRs or TCRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### IV. Polypeptide Expression

The disclosure provides for nucleic acid molecule encoding polypeptides or peptides of the disclosure (e.g TCR genes). These may be generated by methods known in the art, e.g., isolated from B cells of mice that have been immunized and isolated, phage display, expressed in any suitable recombinant expression system and allowed to assemble to form antibody molecules or by recombinant methods.

### A. Expression

The nucleic acid molecules may be used to express large quantities of polypeptides. If the nucleic acid molecules are derived from a non-human, non-transgenic animal, the nucleic acid molecules may be used for humanization of the TCR genes.

### B. Vectors

The disclosure provides for expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, alpha chain, beta chain, or the antigen-binding portion thereof. Expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. A vector that encodes a functionally complete human CH or CL immunoglobulin or TCR sequence may be engineered with appropriate restriction sites so that any variable region sequences can be easily inserted and expressed. A vector that encodes a functionally complete human TCR alpha or TCR beta sequence may be engineered with appropriate restriction sites so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### C. Expression Systems

Numerous expression systems exist that comprise at least a part or all of the expression vectors discussed above. Prokaryote- and/or eukaryote-based systems can be employed for use to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### V. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### A. Host Cells

Also contemplated by the disclosure are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. The antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy, light, alpha, and beta chains of an antibody or TCR, or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### VI. ADDITIONAL THERAPIES

### A. Immunotherapy

The methods may comprise administration of an additional therapy. The additional therapy may comprise a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

The methods of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may include PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may block the CTLA-4 and B7-1 interaction. The inhibitor may block the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

The inhibitor may comprise the heavy and light chain CDRs or VRs of tremelimumab or ipilimumab. Accordingly, the inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may be one that competes for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

### 2. Inhibition of Co-Stimulatory Molecules

The immunotherapy may comprise an inhibitor of a co-stimulatory molecule. The inhibitor may comprise an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv).

Exemplary CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be a particularly suitable chemotherapeutic agent.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

The additional therapy or prior therapy may comprise radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

The additional therapy may comprise surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present disclosure, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### VII. DETECTION AND THERAPEUTIC AGENTS

In methods of the disclosure, it may be useful to detectably or therapeutically label the TCRs or fusion proteins of the disclosure. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

The fluorescent label may be functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

The conjugated agents can be linked to the polypeptide directly or indirectly, using any of a large number of available methods. For example, an agent can be attached at the hinge region of the reduced antibody component via disulfide bond formation, using cross-linkers such as N-succinyl 3-(2-pyridyldithio) proprionate (SPDP), or via a carbohydrate moiety in the Fc region of the antibody (Yu et al., 1994; Upeslacis et al., 1995; Price, 1995).

Techniques for conjugating agents to polypeptides are well known (Amon et al., 1985; Hellstrom et al., 1987; Thorpe, 1985; Baldwin et al., 1985; Thorpe et al., 1982),

The polypeptides of the disclosure or antigen-binding regions thereof can be linked to another functional molecule such as ligands, cytotoxic molecules, chemotherapeutic agents, or other agents described as additional therapeutics.

### VIII. FORMULATIONS AND CULTURE OF THE CELLS

The medium can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

The medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). One or more of these may be explicitly excluded.

The medium may comprise vitamins. The medium may comprise 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. The medium may comprise or consist essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. The vitamins may include or consist essentially of biotin, DL alpha tocopherol acetate, DL alpha-tocopherol, vitamin A, or combinations or salts thereof. The medium may comprise proteins. The proteins may comprise albumin or bovine serum albumin, a fraction of BSA, catalase, insulin, transferrin, superoxide dismutase, or combinations thereof. The medium may comprise one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. The medium may comprise one or more of the following: a B-27® supplement, xeno-free B-27® supplement, GS21TM supplement, or combinations thereof. The medium may comprise or further comprise amino acids, monosaccharides, inorganic ions. The amino acids may comprise arginine, cystine, isoleucine, leucine, lysine, methionine, glutamine, phenylalanine, threonine, tryptophan, histidine, tyrosine, or valine, or combinations thereof. The inorganic ions may comprise sodium, potassium, calcium, magnesium, nitrogen, or phosphorus, or combinations or salts thereof. The medium may comprise one or more of the following: molybdenum, vanadium, iron, zinc, selenium, copper, or manganese, or combinations thereof. The medium may comprise or consist essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. One or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. One or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

The cells of the disclosure may be specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. The cells may be in a formulated cell suspension that is stable at room temperature for 1, 2, 3, or 4 hours or more from time of thawing.

The cells of the disclosure may comprise an engineered TCR, which may be of a defined antigen specificity. The endogenous TCR genes may have been modified by genome editing so that they do not express a protein. Methods of gene editing such as methods using the CRISPR/Cas9 system are known in the art and described herein.

The cells of the disclosure may further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### IX. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). The first and second cancer treatments may be administered in a separate composition. The first and second cancer treatments may be in the same composition.

The cells of the disclosure may be specifically formulated and/or they may be cultured in a particular medium. The cells may be formulated in such a manner as to be suitable for delivery to a recipient without deleterious effects.

The disclosure also relates to compositions and methods comprising therapeutic compositions. The different therapies may be administered in one composition or in more than one composition, such as 2 compositions, 3 compositions, or 4 compositions. Various combinations of the agents may be employed.

The therapeutic compositions of the disclosure may be administered by the same route of administration or by different routes of administration. The cancer therapy may be administered intravenously, intramuscularly, subcutaneously, topically, orally, transdermally, intraperitoneally, intraorbitally, by implantation, by inhalation, intrathecally, intraventricularly, or intranasally. The antibiotic may be administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The cancers amenable for treatment include, but are not limited to, tumors of all types, locations, sizes, and characteristics. The cancer may comprise a solid tumor. The methods provide for a method for reducing tumor volume or treating cancers that are recurrent and/or metastatic. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma, squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### X. Kits

The disclosure also concern kits containing compositions of the disclosure or compositions to implement methods of the invention. Kits can be used to evaluate one or more biomarkers or HLA types. A kit may contain, contain at least or contain at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; a component may be provided individually in the same concentration as it would be in a solution with other components. Concentrations of components may be provided as 1×, 2×, 5×, 10×, or 20× or more.

Negative and/or positive control nucleic acids, probes, and inhibitors may be included in the kits of the disclosure. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

### XI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: TBXT-345 Peptide (SQYPSLWSV-SEQ ID NO: 15) Identification, CTL Generation and TCR-T Development

TBXT (Brachyury) is uniquely expressed in chordoma (about 90% cases), but not any normal tissues. Thus, TBXT is a potential valuable target for chordoma. The inventors identified A0201 restricted peptide (SQYPSLWSV-SEQ ID NO: 15), as well as specific functional T cell receptor-engineered T cells (TCR-T) targeting TBXT. The disclosure provides potential immunotherapeutic approach for the treatment of cancer. Such as peptide could be as therapeutic vaccine, and TCR-T cells can be used as an immunotherapeutic treatment.

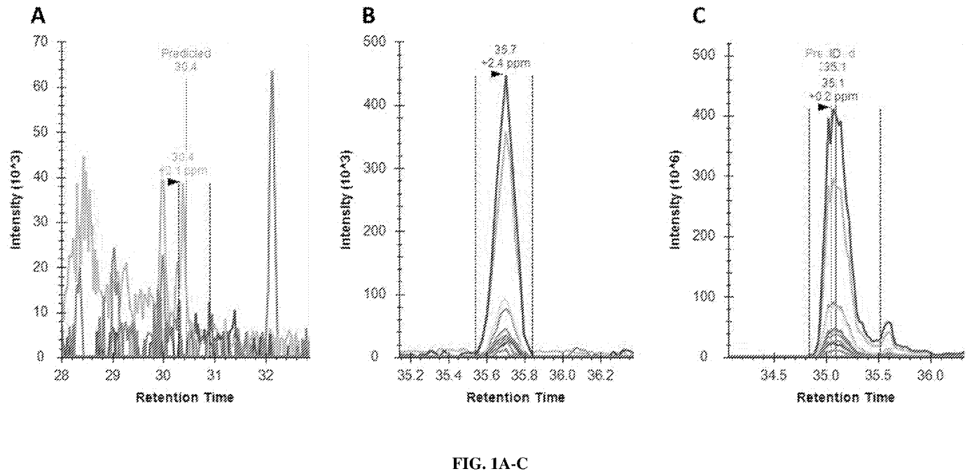

To identify the natural presentation of TBXT-345 in tumor cells, MHC peptide eluted from K562-A2-TBXT (A0201 and TBXT forced expressing) (FIG. 1B), as well as K562-A2-GFP (A0201 and GFP forced expressing, negative control) (FIG. 1A) using an immuno-precipitation (IP) protocol were used for PRM-MS analysis. Pierce™ Peptide Retention Time Calibration Mixture peptides were used to monitor retention time drifts and adjust the scheduled PRM method (FIG. 1C). Synthesized TBXT-345 peptide was spiked into negative control sample (K562-A2-GFP) and analyzed with PRM-MS for the alignment. An inclusion list containing m/z values of protonated precursor peptide ions of interest was generated in Skyline-daily. TBXT-345 peptide peak was detected in K562-A2-TBXT sample (FIG. 1B) and comparable with synthesis peptide (FIG. 1C). No peak was detected in negative sample (FIG. 1A).

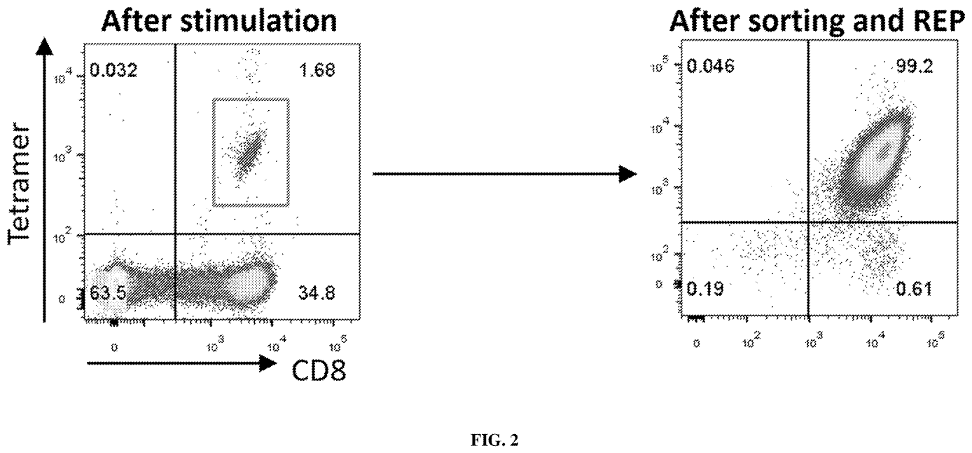

TBXT-345 peptide was pulsed to mature dendritic cells and then co-cultured with autologous PBMC from HLA-A0201 positive healthy donor. After two rounds of stimulation, part of the T cells from the each well were collected for tetramer detection. Then, the tetramer+/CD8+ population were sorted and expanded with rapid expansion protocol (REP). After REP for two weeks, high purity CTL (tetramer+ population over 90%) were observed (FIG. 2).

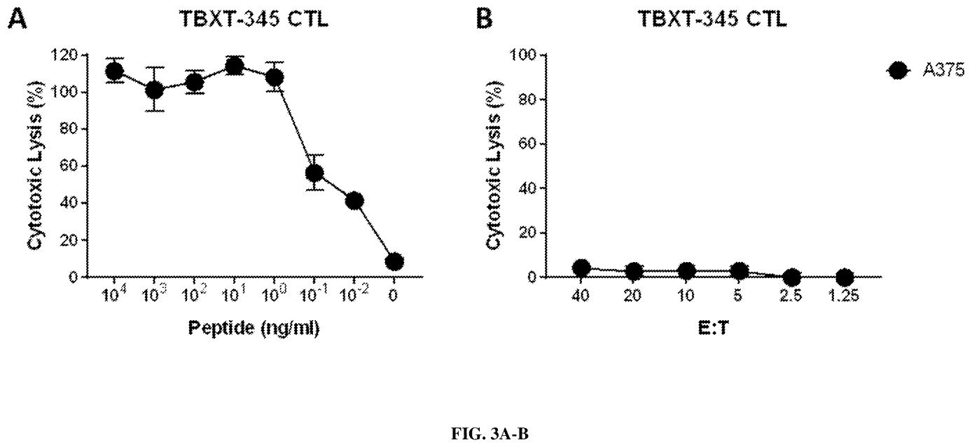

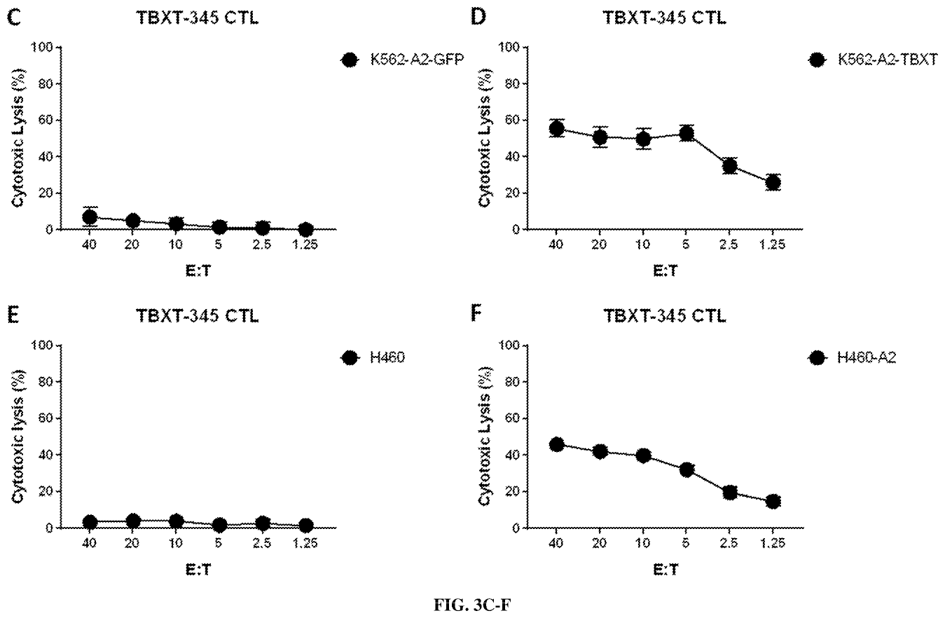

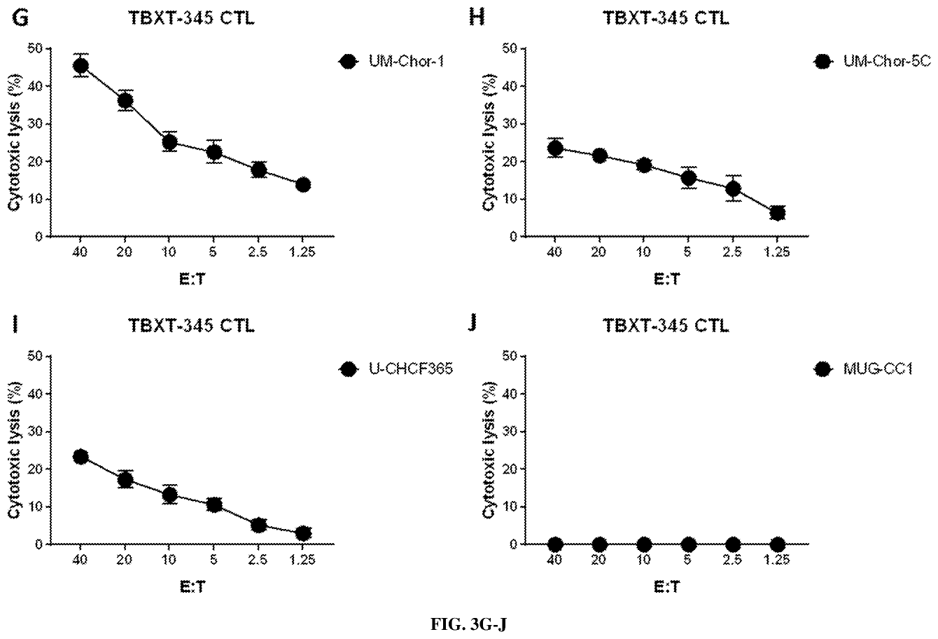

To validate the TBXT-345 specific CTL cell lines, T2 cells pulsed with various concentrations of TBXT-345 peptide were used as targets, and the lysis ability of TBXT-345 CTL cell lines was detected with 4 hour Cr51 release assay (CRA). The effector to target (E:T) ratio used is 20:1. Melanoma cell line A375 (TBXT−, HLA-A0201+) was used as a control cell line FIG. 3B). K562-A2-GFP (A0201+, TBXT−) (FIG. 3C), K562-A2-TBXT (A0201+, TBXT+) (FIG. 3D), H460 (A0201−, TBXT+) (FIG. 3E), and H460-A2 (A0201+, TBXT+) (FIG. 3F) were used as the target to test TBXT−345 CTL recognition using a 4 hour CRA. Chordoma cell lines UM-Chor-1 (FIG. 3G), UM-Chor-5C (FIG. 3H), U-CHCF360 (A0201+, TBXT+) (FIG. 3I), and MUG-CC1 (A0201−, TBXT+) (FIG. 3J) were used as the targets to test TBXT−345 CTL recognition using a 20 hour CRA.

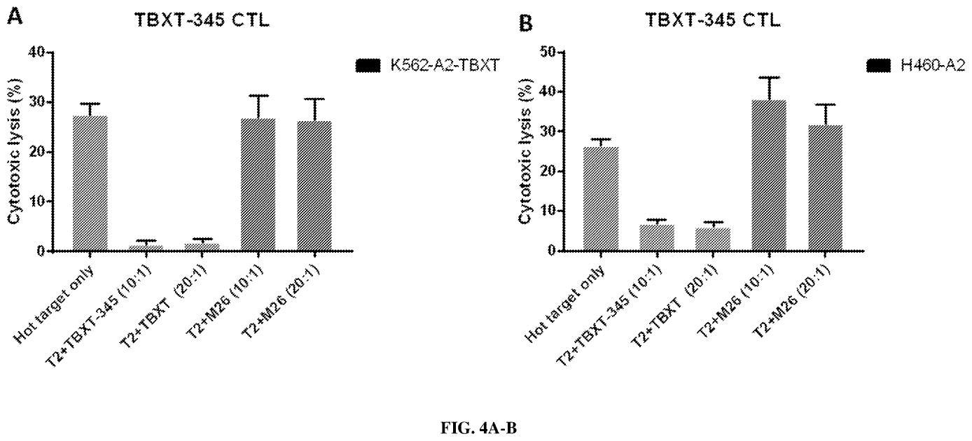

A cold target inhibition assay was used to verify the recognizing specificity of TBXT−345 specific CTL. K562-A2-TBXT (FIG. 4A) and H460-A2 (FIG. 4B) labeled with 51Cr were used as hot targets. The T2 cells pulsed with TBXT−345 peptide without 51Cr labeling were used as cold target. T2 cells pulsed with irrelevant peptide M26 were used as a control cold target. The E:T is 10:1. The cold target: Hot target ratio used is 10:1 or 20:1. The killing inhibition of cold target to TBXT−345 peptide specific CTL was detected with 4 hour CRA (FIG. 4).

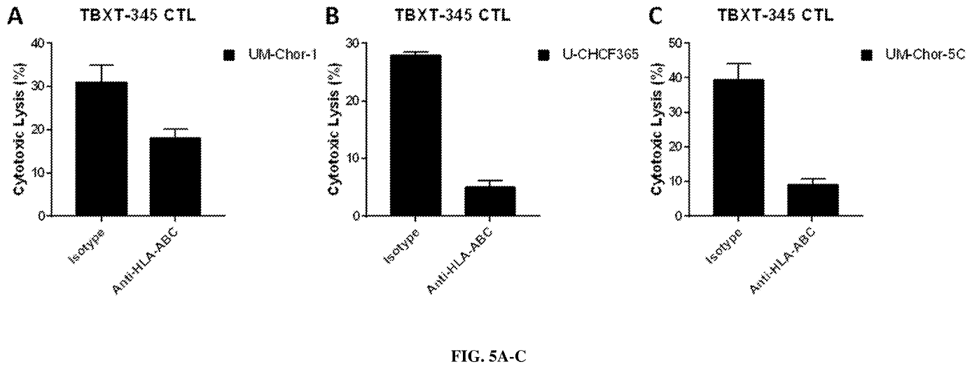

A MHC blocking assay of TBXT−345 specific CTL to chordoma cells with CRA was performed. The Chordoma cell line UM-Chor-1 (FIG. 5A), U-CHCF365 (FIG. 5B) and UM-Chor-5C (FIG. 5C) labeled with 51Cr were co-cultured with TBXT−345 CTL in the presence of anti-HLA-ABC (50 μg/ml) or isotype antibody. The E:T is 20:1. The cytotoxic lysis of TBXT−345 specific CTL to each chordoma cell line was detected with 20 hour CRA.

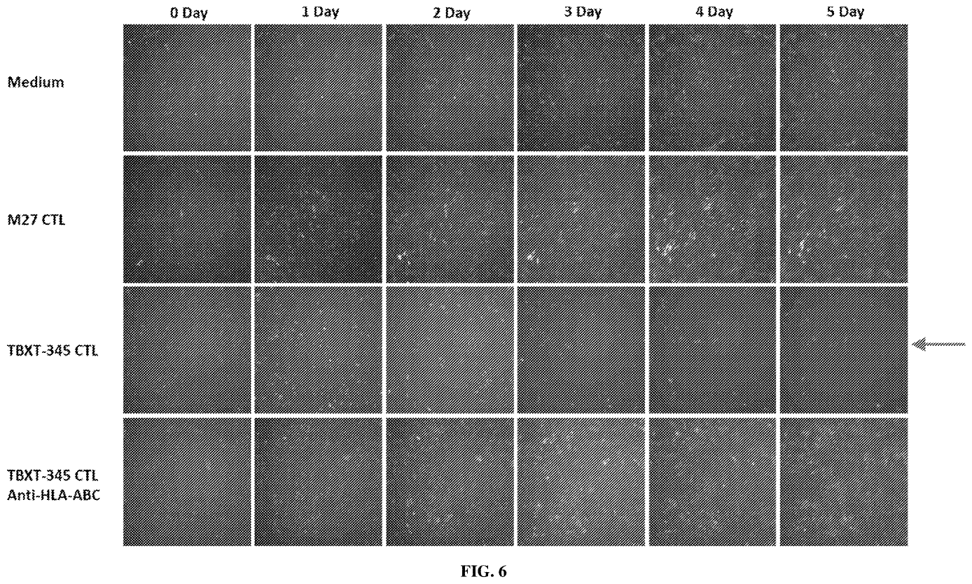

A MHC blocking assay of TBXT−345 specific CTL to chordoma cells with fluorescence detection was performed. GFP expressing chordoma cell line UM-Chor-1 were co-cultured with TBXT−345 CTL in the presence of anti-HLA-ABC (50 μg/ml) or isotype antibody. The E:T is 20:1. Dead cell tracking fluorescence dys Propidium Iodide (PI) was added in the co-culture. The cytotoxic lysis of TBXT−345 specific CTL to chordoma cell line UM-Chor-1 was detected with observing growth of GFP+ cells. GFP-UM-Chor-1 co-cultured with medium only, or M27 irrelevant CTL, were used as a control (FIG. 6).

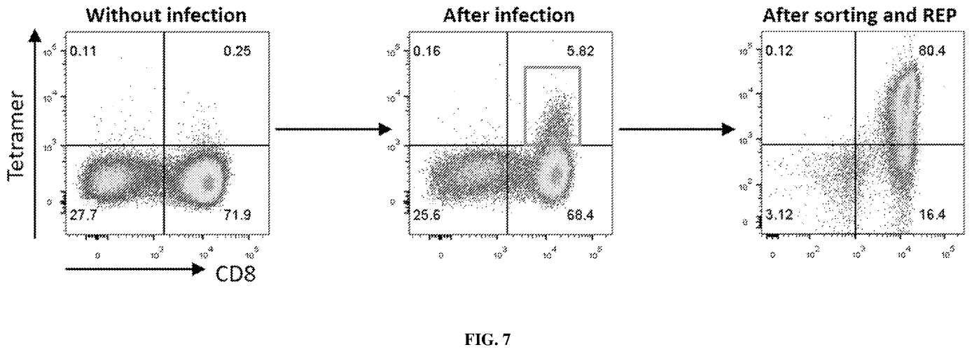

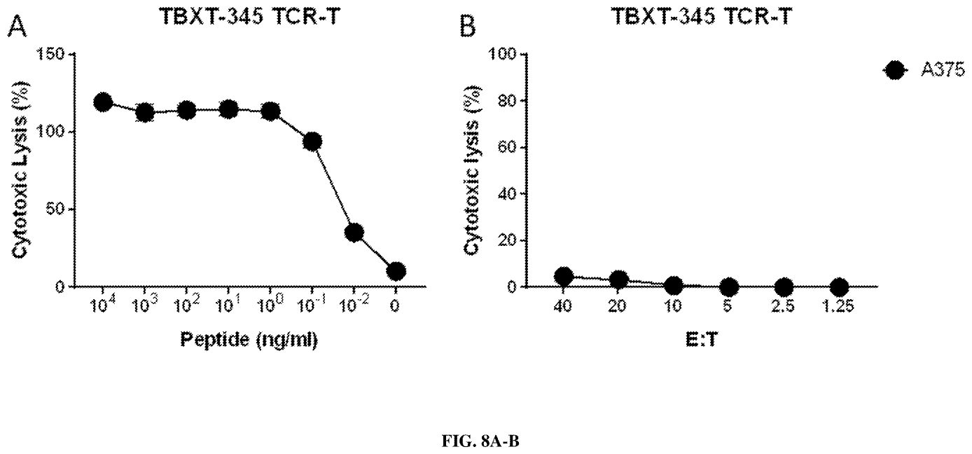

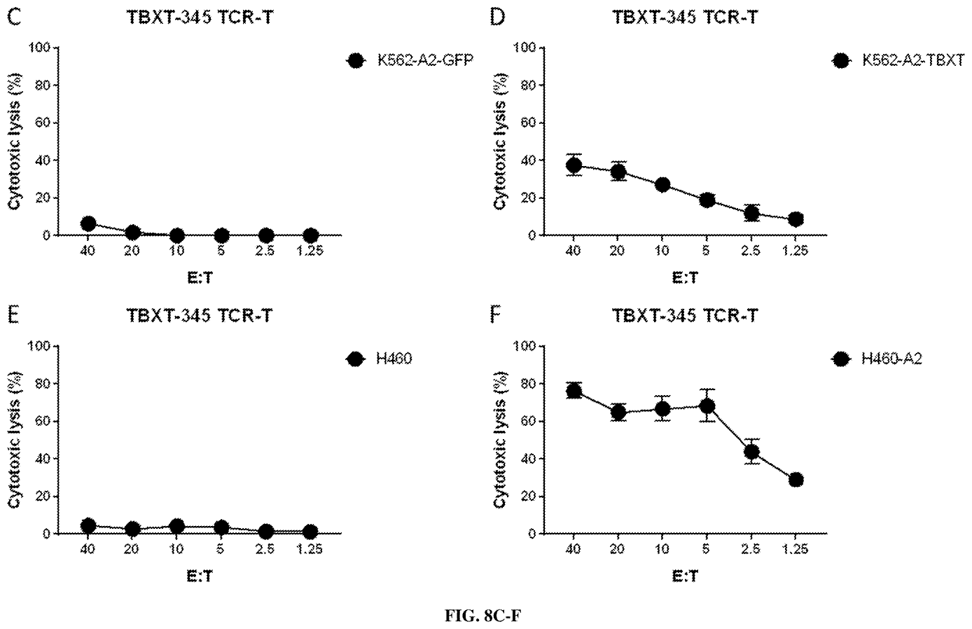

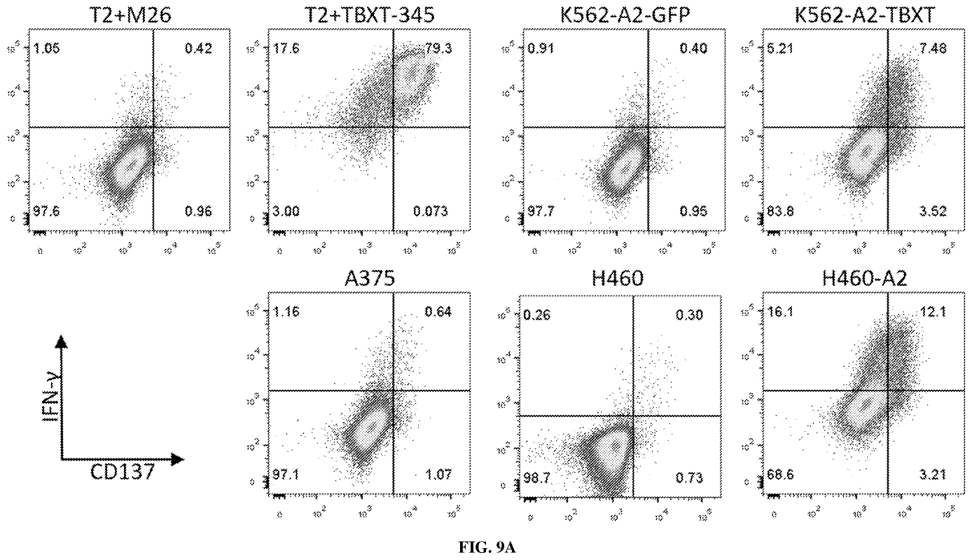

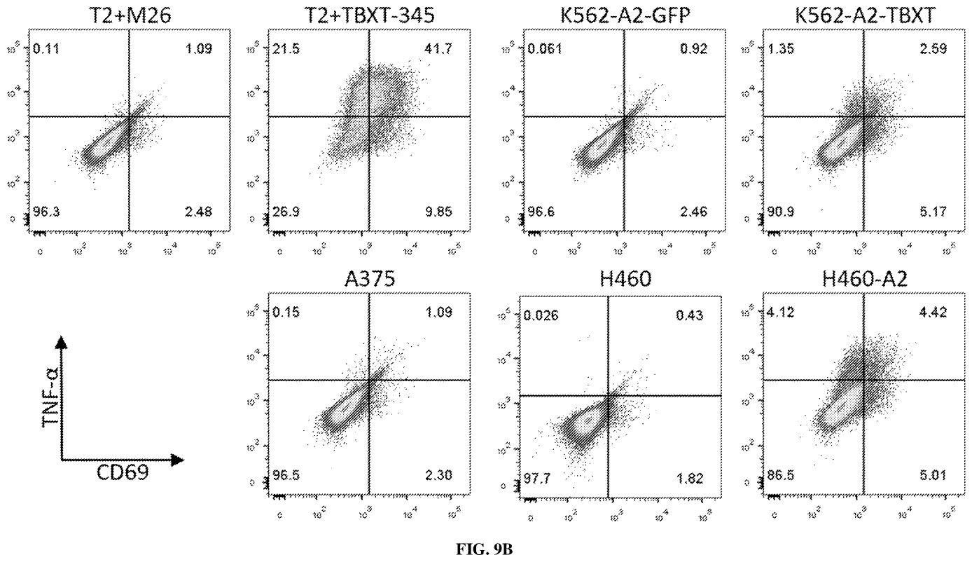

A TBXT−345 specific CTL clonal line (line D12) was selected for TCR cloning and sequencing. TCR alpha chain and beta chain were cloned from TBXT−345 specific CTL D12 line using 5-RACE method. TCR were sequenced and annotated using IMGT tool (IMGT/V-QUEST) (FIG. 7). The whole length of TBXT−345 peptide specific D12 TCR DNA fragment was cloned into retroviral vector pMSGV1 and the recombinant retrovirus was generated. The PBMC from HLA-A0201 healthy donor was infected with recombinant retrovirus. After infection, the tetramer+/CD8+ population was sorted and expanded. After expansion, high purity of TBXT−345 specific D12 TCR-T was obtained (FIG. 7). Functional validation of D12 TCR-T was performed. To test the functionality of the D12 TCRT, T2 cells (FIG. 8A), A375 melanoma cell line (FIG. 8B), K562-A2-GFP (FIG. 8C), K562-A2-TBXT (FIG. 8D), H460 (FIG. 8E), and H460-A2 (FIG. 8F) cells pulsed with various concentrations of TBXT−345 peptide were used as targets, and the lysis ability of TBXT−345 peptide specific D12 TCR-T was detected with 4 hour Cr51 release assay (CRA) (FIG. 8). Next, the inventors used an intracellular cytokine staining assay for the functional detection of TBXT−345 peptide specific D12 TCR-T. The TBXT−345 peptide specific D12 TCR-T cell line was co-cultured with T2+M26 peptide, T2+TBXT−345 peptide, cancer cell line A375, K562-A2-GFP, K562-A2-TBXT, H460, H460-A2 with E:T=10:1 ratio. After overnight co-culturing, the TCR pathway down-stream activated marker, CD137, CD69, IFN-γ and TNF-α were detected with ICS assay (FIG. 9). In FIG. 10, the inventors showed that this D12 T cell line can recognize its cognate epitope and kill tumor cells which express TBXT−345 antigen, but the D12 TCR-T cells did not kill the negative control targets (FIG. 10). The chordoma cell line UM-Chor-1 (FIG. 10A), UM-Chor-5C (FIG. 10B), U-CHCF365 (all are A0201+, TBXT+) (FIG. 10C), and MUC-CC1 (A0201−, TBXT+) (FIG. 10D-control), were used as targets to test the recognition of TBXT-345 peptide specific D12 TCR-T with 20 hour CRA. The effector to target (E:T) ratio is from 40:1 to 1.25:1.

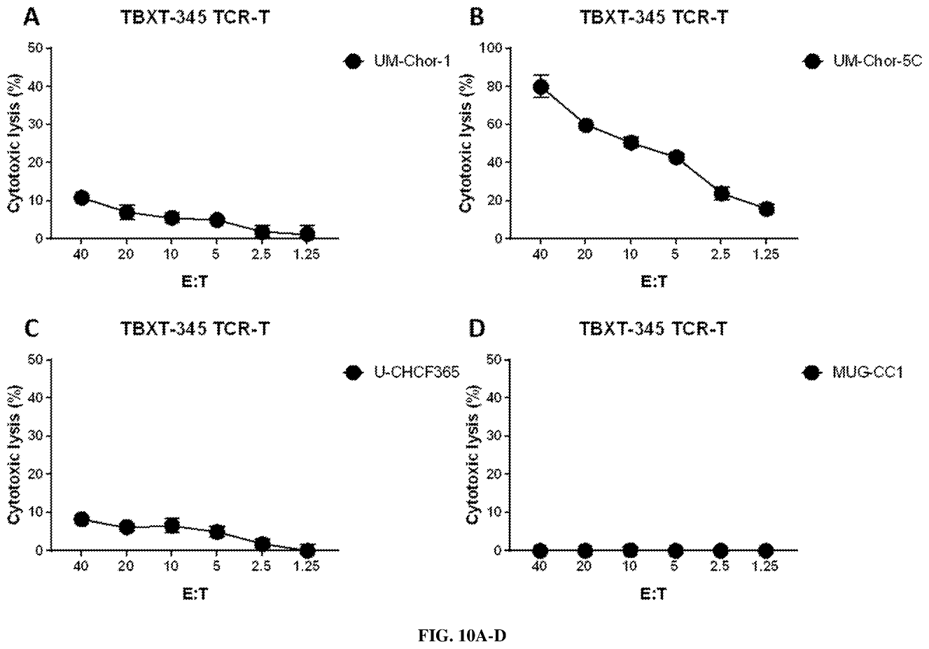

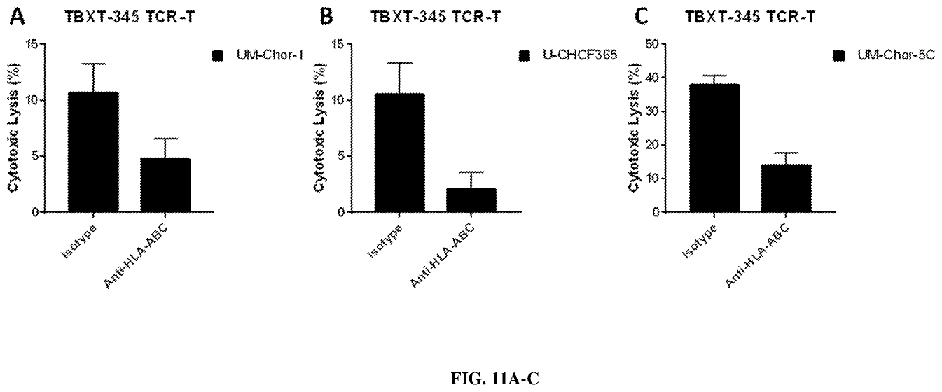

A MHC blocking assay of TBXT−345 specific D12 TCR-T to chordoma cells with CRA was performed. Chordoma cell line UM-Chor-1 (FIG. 11A), U-CHCF365 (FIG. 11B), and UM-Chor-5C (FIG. 11C) labeled with 51Cr were co-cultured with TBXT−345 specific D12 TCR-T in the presence of anti-HLA-ABC (50 μg/ml) or isotype antibody. The E:T is 20:1. The cytotoxic lysis of TBXT−345 specific D12 TCR-T to each chordoma cell line was detected with 20 hour CRA. As shown in FIG. 12, a MHC blocking assay of TBXT−345 D12 TCR-T to chordoma cells with fluorescence detection was performed. GFP expressing chordoma cell line UM-Chor-1 were co-cultured with TBXT−345 TCR-T in the presence of anti-HLA-ABC (50 μg/ml) or isotype antibody. The E:T is 20:1. Dead cell tracking fluorescence dys Propidium Iodide (PI) was added in the co-culture. The cytotoxic lysis of TBXT−345 specific D12 TCR-T to chordoma cell line UM-Chor-1 was detected with observing growth of GFP+ cells. GFP-UM-Chor-1 co-cultured with medium only, or MAGEB2 irrelevant TCR-T, were used as a control.

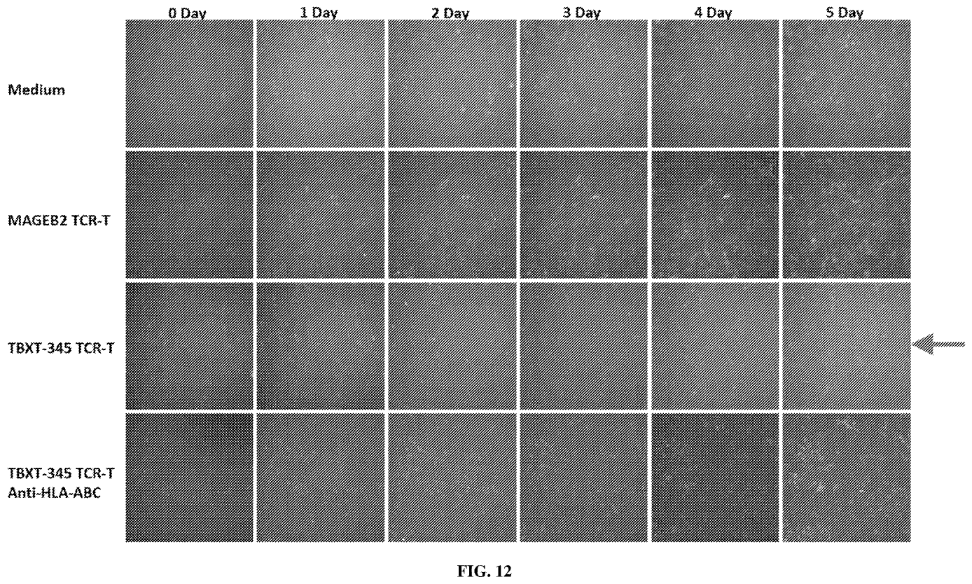

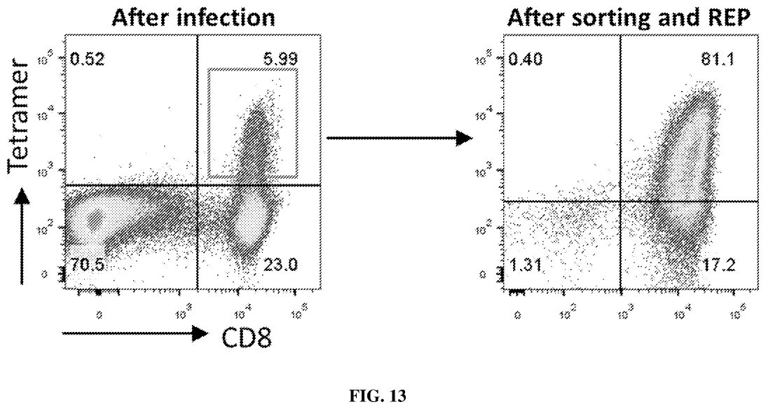

A second TBXT−345 specific CTL clonal cell line (line B4) was selected for TCR cloning and sequencing. TCR alpha chain and beta chain were cloned from the TBXT−345 specific CTL B4 clone using 5-RACE method. The TCR chains were sequenced and annotated using IMGT tool (IMGT/V-QUEST). The whole length of TBXT−345 B4 clone specific TCR DNA fragment was cloned into retroviral vector pMSGV1 and the recombinant retrovirus was generated. The PBMC from HLA-A0201 healthy donor was infected with recombinant retrovirus. After infection, the tetramer+/CD8+ population was sorted and expanded. After expansion, high purity of TBXT−345 specific B4 TCR-T was obtained (FIG. 13).

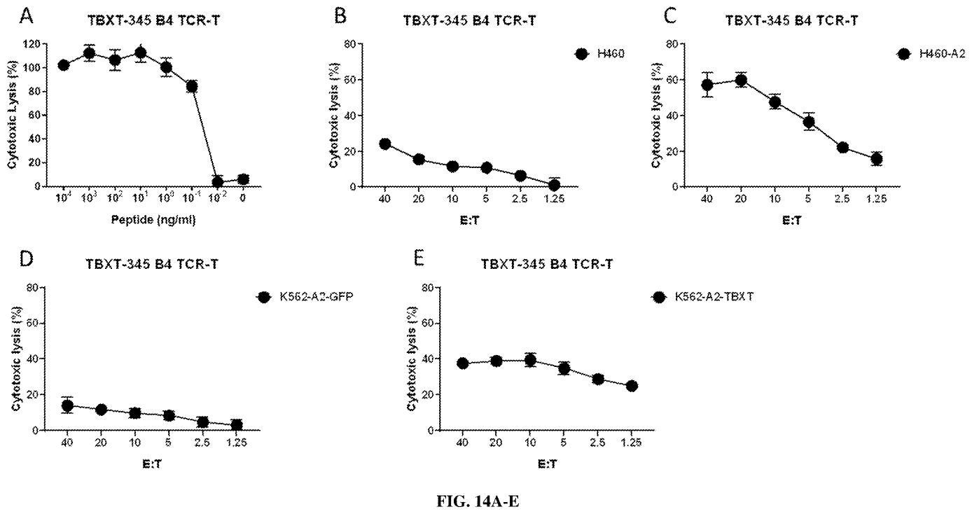

Functional validation of B4 TCR-T was performed, as shown in FIG. 14A-E. To test the functionality of the TCR-T, T2 cells pulsed with various concentrations of TBXT−345 peptide were used as targets (FIG. 14A). The lysis ability of TBXT−345 B4 TCR-T was detected with 4 hour Cr51 release assay (CRA). The effector to target (E:T) ratio was 20:1. Lung cancer cell lines H460 (FIG. 14B; A0201−, TBXT+), and H460-A2 (FIG. 14C; A0201+, TBXT+), as well as leukemia cell lines K562-A2-GFP (FIG. 14D; A0201+, TBXT−), K562-A2-TBXT (FIG. 14E; A0201+, TBXT+), were used as targets to test the recognition of TBXT-345 B4 TCR-T with 4 hour CRA. The effector to target (E:T) ratio ranged from 40:1 to 1.25:1.

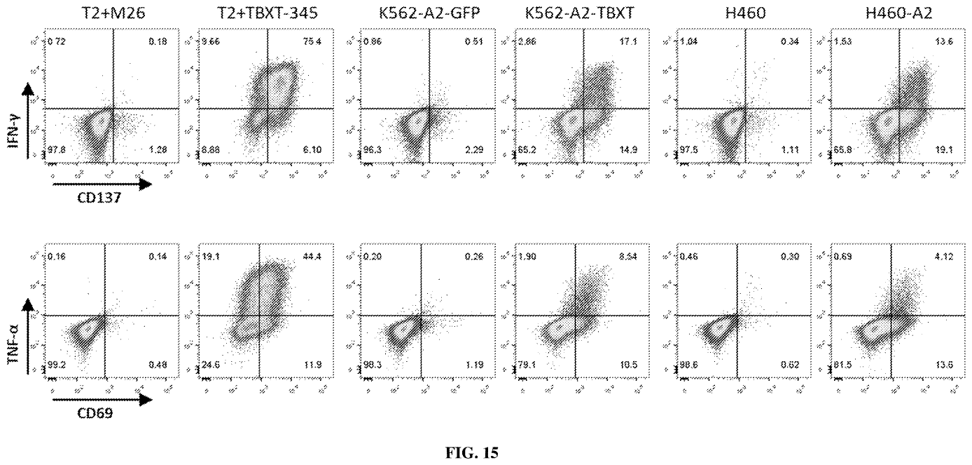

The inventors used intracellular cytokine staining (ICS) assay for functional assessment of the TBXT−345 B4 TCR-T in the presence of various peptides and cell lines, as shown in FIG. 15. TBXT−345 B4 TCR-T cells were co-cultured with T2+M26 peptide, T2+TBXT−345 peptide, or cancer cell line A375, K562-A2-GFP, K562-A2-TBXT, H460, or H460-A2 with E:T=10:1 ratio. After overnight co-culturing, the TCR pathway down-stream activation markers CD137, CD69, IFN-γ and TNF-α were detected with ICS assay and flow cytometry (FIG. 15).

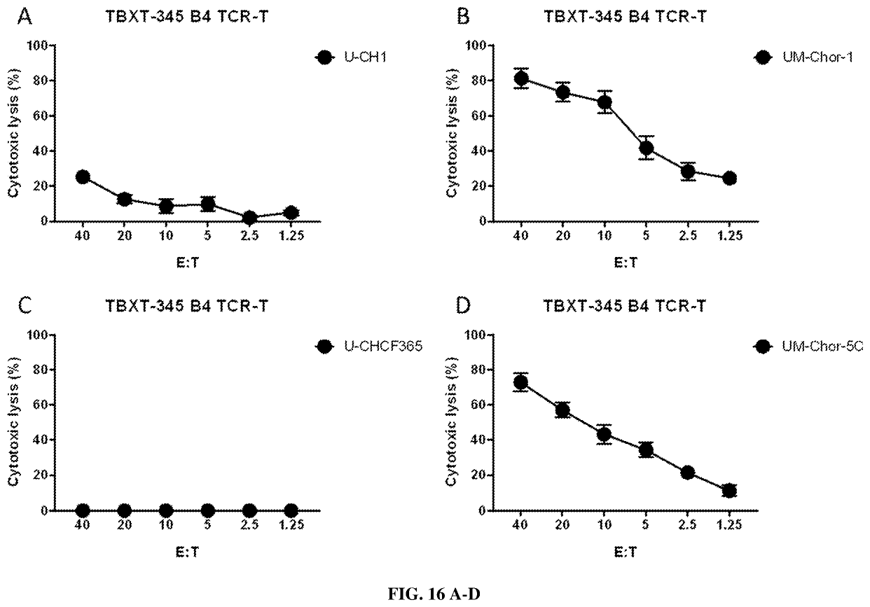

The inventors validated recognition and killing of chordoma cell lines by TBXT−345 B4 TCR-T cells, as shown in FIG. 16. Chordoma cell lines U-CH1 (FIG. 16A), UM-Chor-1 (FIG. 16B), U-CHCF365 (FIG. 16C), UM-Chor-5C (FIG. 16D) (all are A0201+, TBXT+), were as targets to test the recognition of TBXT−345 B4 TCR-T with 20 hour CRA. The effector to target (E:T) ratio ranged from 40:1 to 1.25:1.

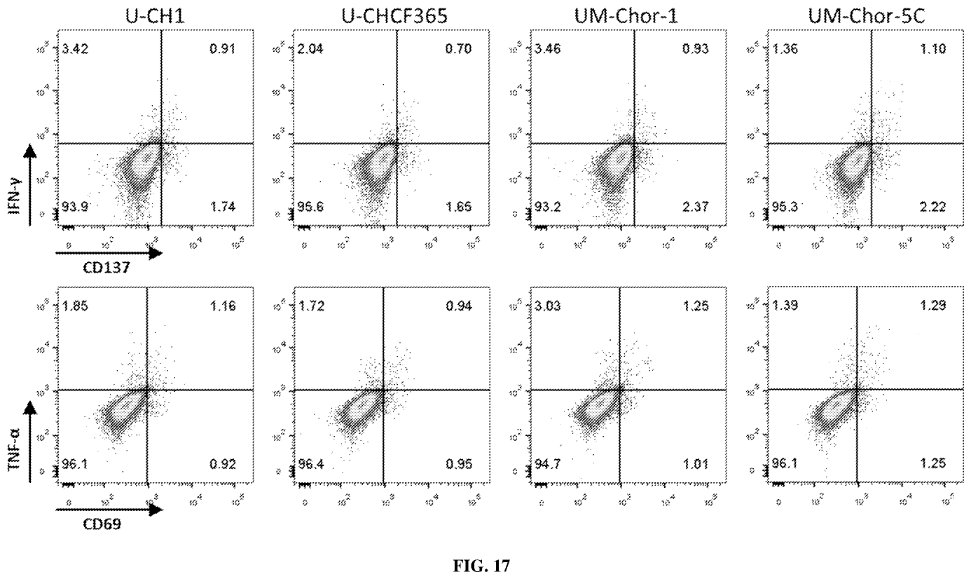

The inventors used intracellular cytokine staining (ICS) assay for functional assessment of the TBXT−345 B4 TCR-T in the presence of chordoma cell lines, as shown in FIG. 17. The TBXT−345 B4 TCR-T cells were co-cultured with chordoma cell lines U-CH1, U-CHCF365, UM-Chor-1 and UM-Chor-5C with E:T=10:1 ratio. After overnight co-culturing, the TCR pathway down-stream activation markers CD137, CD69, IFN-γ and TNF-α were detected with ICS assay and flow cytometry (FIG. 17).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polypeptide comprising an antigen binding variable region comprising a CDR3 comprising an amino acid sequence with at least 80% sequence identity to SEQ ID NO: 8.

2. The polypeptide of claim 1, wherein the CDR3 comprises the amino acid sequence of SEQ ID NO: 8.

3. The polypeptide of claim 1 or 2, wherein the variable region comprises a CDR1, CDR2, and/or CDR3.

4. The polypeptide of claim 3, wherein the variable region comprises a CDR1 with at least 80% sequence identity to SEQ ID NO: 6.

5. The polypeptide of claim 3 or 4, wherein the variable region comprises a CDR2 with at least 80% sequence identity to SEQ ID NO: 7.

6. The polypeptide of claim 4 or 5, wherein the variable region comprises a CDR1 comprising the amino acid sequence of SEQ ID NO: 6 and/or a CDR2 comprising the amino acid sequence of SEQ ID NO: 7.

7. The polypeptide of any one of claims 1-6, wherein the variable region comprises an amino acid sequence with at least 70% sequence identity to SEQ ID NO: 4.

8. The polypeptide of claim 7, wherein the variable region comprises the amino acid sequence of SEQ ID NO: 4.

9. The polypeptide of any one of claims 1-8, wherein the polypeptide comprises a T cell receptor alpha (TCR-a) variable region.

10. The polypeptide of claim 9, wherein the polypeptide comprises a TCR-a variable and constant region.

11. The polypeptide of any one of claims 1-10, wherein the polypeptide further comprises a signal peptide.

12. The polypeptide of claim 11, wherein the signal peptide comprises an amino acid sequence with at least 80% identity to SEQ ID NO: 5.

13. The polypeptide of claim 12, wherein the signal peptide comprises an amino acid sequence of SEQ ID NO: 5.

14. A polypeptide comprising an antigen binding variable region comprising a CDR3 comprising an amino acid sequence with at least 80% sequence identity to SEQ ID NO: 14.

15. The polypeptide of claim 14, wherein the CDR3 comprises the amino acid sequence of SEQ ID NO: 14.

16. The polypeptide of claim 14 or 15, wherein the variable region comprises a CDR1, CDR2, and/or CDR3.

17. The polypeptide of claim 16, wherein the variable region comprises a CDR1 with at least 80% sequence identity to SEQ ID NO: 12.

18. The polypeptide of claim 16 or 17, wherein the variable region comprises a CDR2 with at least 80% sequence identity to SEQ ID NO: 13.

19. The polypeptide of claim 17 or 18, wherein the variable region comprises a CDR1 comprising the amino acid sequence of SEQ ID NO: 12 and/or a CDR2 comprising the amino acid sequence of SEQ ID NO: 13.

20. The polypeptide of any one of claims 14-19, wherein the variable region comprises an amino acid sequence with at least 70% sequence identity to SEQ ID NO: 10.

21. The polypeptide of claim 20, wherein the variable region comprises the amino acid sequence of SEQ ID NO: 10.

22. The polypeptide of any one of claims 14-21, wherein the polypeptide comprises a T cell receptor beta (TCR-b) variable region.

23. The polypeptide of claim 22, wherein the polypeptide comprises a TCR-b variable and constant region.

24. The polypeptide of any one of claims 14-23, wherein the polypeptide further comprises a signal peptide.

25. The polypeptide of claim 24, wherein the signal peptide comprises an amino acid sequence with at least 80% identity to SEQ ID NO: 11.

26. The polypeptide of claim 25, wherein the signal peptide comprises an amino acid sequence of SEQ ID NO: 11.

27. An engineered T-cell Receptor (TCR) comprising a TCR-a polypeptide and a TCR-b polypeptide, wherein the TCR-a polypeptide comprises a CDR3 comprising an amino acid sequence with at least 80% sequence identity to SEQ ID NO: 8 and the TCR-b polypeptide comprises a CDR3 comprising an amino acid sequence with at least 80% sequence identity to SEQ ID NO: 14.

28. The TCR of claim 27, wherein the TCR-a polypeptide comprises a CDR3 comprising the amino acid sequence of SEQ ID NO: 8 and the TCR-b polypeptide comprises a CDR3 comprising the amino acid sequence of SEQ ID NO: 14.

29. The TCR of claim 27 or 28, wherein the TCR comprises a TCR-a polypeptide comprising a variable region comprising CDR1, CDR2, and CDR3 and a TCR-b polypeptide comprising a variable region comprising CDR1, CDR2, and CDR3.

30. The TCR of claim 29, wherein the TCR-a polypeptide comprises a CDR1 with at least 80% sequence identity to SEQ ID NO: 6 and/or the TCR-b polypeptide comprises a CDR1 with at least 80% sequence identity to SEQ ID NO: 12.

31. The TCR of claim 30, wherein the TCR-a polypeptide comprises a CDR1 comprising the amino acid sequence of SEQ ID NO: 6 and the TCR-b polypeptide comprises a CDR1 comprising the amino acid sequence of SEQ ID NO: 12.

32. The TCR of any one of claims 29-31, wherein the TCR-a polypeptide comprises a CDR2 with at least 80% sequence identity to SEQ ID NO: 7 and the TCR-b polypeptide comprises a CDR2 with at least 80% sequence identity to SEQ ID NO: 13.

33. The TCR of claim 32, wherein the TCR-a polypeptide comprises a CDR2 comprising the amino acid sequence of SEQ ID NO: 7 and the TCR-b polypeptide comprises a CDR2 comprising the amino acid sequence of SEQ ID NO: 13.

34. The TCR of any one of claims 29-33, wherein the CDR1, CDR2, and CDR3 of the TCR-a polypeptide comprise the amino acid sequence of SEQ ID NO: 6, 7, and 8, respectively and wherein the CDR1, CDR3, and CDR3 of the TCR-b polypeptide comprise the amino acid sequence of SEQ ID NO: 12, 13, and 14, respectively.

35. The TCR of any one of claims 29-34, wherein the TCR-a polypeptide comprises an amino acid sequence with at least 70% sequence identity to SEQ ID NO: 4 and the TCR-b polypeptide comprises an amino acid sequence with at least 70% sequence identity to SEQ ID NO: 10.

36. The TCR of claim 35, wherein the TCR-a polypeptide comprises the amino acid sequence of SEQ ID NO: 4 and the TCR-b polypeptide comprises an amino acid sequence with at least 70% sequence identity to SEQ ID NO: 10.

37. The TCR of any one of claims 27-36, wherein the TCR comprises a modification or is chimeric.

38. The TCR of any one of claims 27-37, wherein the TCR-a polypeptide and TCR-b polypeptide are operably linked.

39. The TCR of claim 38, wherein the TCR-a polypeptide and TCR-b polypeptide are operably linked through a peptide bond.

40. The TCR of claim 38, wherein the TCR is a single chain TCR.

41. The TCR of claim 39 or 40, wherein the TCR-a polypeptide and TCR-b polypeptide are on the same polypeptide and wherein the TCR-b is amino-proximal to the TCR-a.

42. The TCR of claim 39 or 40, wherein the TCR-a polypeptide and TCR-b polypeptide are on the same polypeptide and wherein the TCR-a is amino-proximal to the TCR-b.

43. The TCR of any one of claims 39-42, wherein the TCR comprises a linker between the TCR-a and TCR-b polypeptide.

44. The TCR of any one of claims 39-43, wherein the linker comprises glycine and serine residues.

45. A polypeptide comprising an antigen binding variable region comprising a CDR3 comprising an amino acid sequence with at least 80% sequence identity to SEQ ID NO: 35.

46. The polypeptide of claim 45, wherein the CDR3 comprises the amino acid sequence of SEQ ID NO: 35.

47. The polypeptide of claim 45 or 46, wherein the variable region comprises a CDR1, CDR2, and/or CDR3.

48. The polypeptide of claim 47, wherein the variable region comprises a CDR1 with at least 80% sequence identity to SEQ ID NO: 33.

49. The polypeptide of claim 47 or 48, wherein the variable region comprises a CDR2 with at least 80% sequence identity to SEQ ID NO: 34.

50. The polypeptide of claim 48 or 49, wherein the variable region comprises a CDR1 comprising the amino acid sequence of SEQ ID NO: 33 and/or a CDR2 comprising the amino acid sequence of SEQ ID NO: 34.

51. The polypeptide of any one of claims 45-50, wherein the variable region comprises an amino acid sequence with at least 70% sequence identity to SEQ ID NO: 31.

52. The polypeptide of claim 51, wherein the variable region comprises the amino acid sequence of SEQ ID NO: 31.

53. The polypeptide of any one of claims 45-52, wherein the polypeptide comprises a T cell receptor alpha (TCR-a) variable region.

54. The polypeptide of claim 53, wherein the polypeptide comprises a TCR-a variable and constant region.

55. The polypeptide of any one of claims 45-54, wherein the polypeptide further comprises a signal peptide.

56. The polypeptide of claim 55, wherein the signal peptide comprises an amino acid sequence with at least 80% identity to SEQ ID NO: 32.

57. The polypeptide of claim 56, wherein the signal peptide comprises an amino acid sequence of SEQ ID NO: 32.

58. A polypeptide comprising an antigen binding variable region comprising a CDR3 comprising an amino acid sequence with at least 80% sequence identity to SEQ ID NO: 41.

59. The polypeptide of claim 58, wherein the CDR3 comprises the amino acid sequence of SEQ ID NO: 41.

60. The polypeptide of claim 58 or 59, wherein the variable region comprises a CDR1, CDR2, and/or CDR3.

61. The polypeptide of claim 60, wherein the variable region comprises a CDR1 with at least 80% sequence identity to SEQ ID NO: 39.

62. The polypeptide of claim 60 or 61, wherein the variable region comprises a CDR2 with at least 80% sequence identity to SEQ ID NO: 40.

63. The polypeptide of claim 61 or 62, wherein the variable region comprises a CDR1 comprising the amino acid sequence of SEQ ID NO: 39 and/or a CDR2 comprising the amino acid sequence of SEQ ID NO: 40.

64. The polypeptide of any one of claims 58-63, wherein the variable region comprises an amino acid sequence with at least 70% sequence identity to SEQ ID NO: 37.

65. The polypeptide of claim 64, wherein the variable region comprises the amino acid sequence of SEQ ID NO: 37.

66. The polypeptide of any one of claims 58-65, wherein the polypeptide comprises a T cell receptor beta (TCR-b) variable region.

67. The polypeptide of claim 66, wherein the polypeptide comprises a TCR-b variable and constant region.

68. The polypeptide of any one of claims 58-67, wherein the polypeptide further comprises a signal peptide.

69. The polypeptide of claim 68, wherein the signal peptide comprises an amino acid sequence with at least 80% identity to SEQ ID NO: 38.

70. The polypeptide of claim 69, wherein the signal peptide comprises an amino acid sequence of SEQ ID NO: 38.

71. An engineered T-cell Receptor (TCR) comprising a TCR-a polypeptide and a TCR-b polypeptide, wherein the TCR-a polypeptide comprises a CDR3 comprising an amino acid sequence with at least 80% sequence identity to SEQ ID NO: 35 and the TCR-b polypeptide comprises a CDR3 comprising an amino acid sequence with at least 80% sequence identity to SEQ ID NO: 41.

72. The TCR of claim 71, wherein the TCR-a polypeptide comprises a CDR3 comprising the amino acid sequence of SEQ ID NO: 35 and the TCR-b polypeptide comprises a CDR3 comprising the amino acid sequence of SEQ ID NO: 41.

73. The TCR of claim 71 or 72, wherein the TCR comprises a TCR-a polypeptide comprising a variable region comprising CDR1, CDR2, and CDR3 and a TCR-b polypeptide comprising a variable region comprising CDR1, CDR2, and CDR3.

74. The TCR of claim 73, wherein the TCR-a polypeptide comprises a CDR1 with at least 80% sequence identity to SEQ ID NO: 33 and/or the TCR-b polypeptide comprises a CDR1 with at least 80% sequence identity to SEQ ID NO: 39.

75. The TCR of claim 74, wherein the TCR-a polypeptide comprises a CDR1 comprising the amino acid sequence of SEQ ID NO: 33 and the TCR-b polypeptide comprises a CDR1 comprising the amino acid sequence of SEQ ID NO: 39.

76. The TCR of any one of claims 73-75, wherein the TCR-a polypeptide comprises a CDR2 with at least 80% sequence identity to SEQ ID NO: 34 and the TCR-b polypeptide comprises a CDR2 with at least 80% sequence identity to SEQ ID NO: 40.

77. The TCR of claim 76, wherein the TCR-a polypeptide comprises a CDR2 comprising the amino acid sequence of SEQ ID NO: 34 and the TCR-b polypeptide comprises a CDR2 comprising the amino acid sequence of SEQ ID NO: 40.

78. The TCR of any one of claims 73-77, wherein the CDR1, CDR2, and CDR3 of the TCR-a polypeptide comprise the amino acid sequence of SEQ ID NO: 33, 34, and 35, respectively and wherein the CDR1, CDR3, and CDR3 of the TCR-b polypeptide comprise the amino acid sequence of SEQ ID NO: 39, 40, and 41, respectively.

79. The TCR of any one of claims 73-78, wherein the TCR-a polypeptide comprises an amino acid sequence with at least 70% sequence identity to SEQ ID NO: 31 and the TCR-b polypeptide comprises an amino acid sequence with at least 70% sequence identity to SEQ ID NO: 37.

80. The TCR of claim 79, wherein the TCR-a polypeptide comprises the amino acid sequence of SEQ ID NO: 31 and the TCR-b polypeptide comprises an amino acid sequence with at least 70% sequence identity to SEQ ID NO: 37.

81. The TCR of any one of claims 71-80, wherein the TCR comprises a modification or is chimeric.

82. The TCR of any one of claims 71-81, wherein the TCR-a polypeptide and TCR-b polypeptide are operably linked.

83. The TCR of claim 82, wherein the TCR-a polypeptide and TCR-b polypeptide are operably linked through a peptide bond.

84. The TCR of claim 82, wherein the TCR is a single chain TCR.

85. The TCR of claim 83 or 84, wherein the TCR-a polypeptide and TCR-b polypeptide are on the same polypeptide and wherein the TCR-b is amino-proximal to the TCR-a.

86. The TCR of claim 83 or 84, wherein the TCR-a polypeptide and TCR-b polypeptide are on the same polypeptide and wherein the TCR-a is amino-proximal to the TCR-b.

87. The TCR of any one of claims 83-86, wherein the TCR comprises a linker between the TCR-a and TCR-b polypeptide.

88. The TCR of any one of claims 83-87, wherein the linker comprises glycine and serine residues.

89. A peptide comprising at least 66% sequence identity to SEQ ID NO: 15; wherein the peptide is less than 100 amino acids in length.

90. The peptide of claim 89, wherein the peptide comprises SEQ ID NO: 15.

91. The peptide of claim 89, wherein the peptide comprises at least 6 contiguous amino acids of SEQ ID NO: 15.

92. The peptide of claim 89, wherein the peptide comprises at least 7 contiguous amino acids of SEQ ID NO: 15.

93. The peptide of claim 89, wherein the peptide comprises at least 8 contiguous amino acids of SEQ ID NO: 15.

94. The peptide of any one of claims 89-93, wherein the peptide comprises at least 77% sequence identity to SEQ ID NO: 15.

95. The peptide of any one of claims 89-94, wherein the peptide comprises at least 88% sequence identity to SEQ ID NO: 15.

96. The peptide of any one of claims 89-95, wherein the peptide consists of 7 amino acids.

97. The peptide of any one of claims 89-95, wherein the peptide consists of 8 amino acids.

98. The peptide of any one of claims 89-95, wherein the peptide consists of 9 amino acids.

99. The peptide of claim 89, wherein the peptide consists of SEQ ID NO: 15.

100. The peptide of any one of claims 89-99, wherein the peptide is immunogenic.

101. The peptide of any one of claims 89-100, wherein the peptide is modified.

102. The peptide of claim 101, wherein the modification comprises conjugation to a molecule.

103. The peptide of claim 101 or 102, wherein the molecule comprises an antibody, a lipid, an adjuvant, or a detection moiety.

104. The peptide of any of claims 89-103, wherein the peptide has 1, 2 or 3 substitutions relative to a peptide of SEQ ID NO: 15.

105. A polypeptide comprising the peptide of any one of claims 89-104.

106. A composition comprising at least one MHC polypeptide and the peptide or polypeptide of any one of claims 89-105.

107. The composition of claim 106, wherein the MHC polypeptide is and/or peptide is conjugated to a detection tag.

108. The composition of claim 106 or 107, wherein the MHC polypeptide and peptide are operatively linked.

109. The composition of claim 108, wherein the MHC polypeptide and peptide are operatively linked through a peptide bond.

110. The composition of claim 108, wherein the MHC polypeptide and peptide are operatively linked through van der Waals forces.

111. The composition of any one of claims 106-110, wherein at least two MHC polypeptides are linked to one peptide.

112. The composition of any one of claims 106-111, wherein the average ratio of MHC polypeptides to peptides is 4:1.

113. A molecular complex comprising the peptide or polypeptide of any one of claims 89-105 and a MHC polypeptide.

114. A peptide-specific binding molecule, wherein the molecule specifically binds to a peptide or polypeptide of any one of claim 89-105 or the molecular complex of claim 113.

115. The binding molecule of claim 114, wherein the binding molecule is an antibody, TCR mimc antibody, scFV, nanobody, aptamer, or DARPIN.

116. A method of producing peptide-specific immune effector cells comprising: contacting a starting population of immune effector cells with a peptide or polypeptide of any one of claims 89-105 or the molecular complex of claim 113, thereby generating peptide-specific immune effector cells.

117. The method of claim 116, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

118. The method of claim 117, wherein the APCs are dendritic cells.

119. The method of any one of claims 116-118, wherein the immune effector cells are T cells, peripheral blood lymphocytes, NK cells, invariant NK cells, NKT cells.

120. The method of any one of claims 116-119, wherein the immune effector cells have been differentiated from mesenchymal stem cell (MSC) or induced pluripotent stem (iPS) cells.

121. The method of claim 120, wherein the T cells are CD8⁺ T cells, CD4⁺ T cells, or γδ T cells.

122. The method of claim 120, wherein the T cells are cytotoxic T lymphocytes (CTLs).

123. The method of any one of claims 116-122, wherein the method comprises isolating the starting population of immune effector cells from peripheral blood mononuclear cells (PBMCs).

124. The method of any one of claims 116-123, wherein the starting population of immune effector cells is obtained from a subject.

125. The method of claim 124, wherein the subject is a human.

126. The method of claim 124 or 125, wherein the subject has cancer.

127. The method of claim 126, wherein the cancer comprises a TBXT antigen positive cancer.

128. The method of claim 127, wherein the cancer comprises cancer cells that are positive for a peptide of SEQ ID NO: 15.

129. The method of any one of claims 126-128, wherein the subject has been determined to have a TBXT antigen positive cancer.

130. The method of claim 129, wherein the subject has been determined to have cancer cells that are positive for a peptide of SEQ ID NO: 15.

131. The method of any one of claims 126-130, wherein the subject has been diagnosed with the cancer.

132. The method of any one of claims 126-131, wherein the cancer comprises chordoma.

133. The method of any one of claims 126-132, wherein the cancer comprises small cell lung cancer.

134. The method of any one of claims 116-133, wherein the method further comprises introducing the peptide or a nucleic acid encoding the peptide into the dendritic cells prior to the co-culturing.

135. The method of claim 134, where the peptide or nucleic acids encoding the peptide are introduced by electroporation.

136. The method of claim 134, wherein the peptide or nucleic acids encoding the peptide are introduced by adding the peptide or nucleic acid encoding the peptide to the dendritic cell culture media.

137. The method of any one of claims 116-136, wherein the immune effector cells are co-cultured with a second population of dendritic cells into which the peptide or the nucleic acid encoding the peptide has been introduced.

138. The method of any one of claims 116-137, wherein a population of CD8 or CD4-positive and peptide MHC tetramer-positive T cells are purified from the immune effector cells following the co-culturing.

139. The method of claim 138, wherein a clonal population of peptide-specific immune effector cells are generated by limiting or serial dilution followed by expansion of individual clones by a rapid expansion protocol.

140. The method of claim 139, wherein the method further comprises cloning of a T cell receptor (TCR) from the clonal population of peptide-specific immune effector cells.

141. The method of claim 140, wherein cloning of the TCR is cloning of a TCR alpha and a beta chain.

142. The method of claim 140 or 141, wherein the TCR is cloned using a 5′-Rapid amplification of cDNA ends (RACE) method.

143. The method of claim 142, wherein the cloned TCR is subcloned into an expression vector.

144. The method of claim 143, wherein the expression vector is a retroviral or lentiviral vector.

145. The method of claim 143 or 144, where the method further comprises transducing a host cell with the expression vector to generate an engineered cell that expresses the TCR.

146. The method of claim 145, wherein the host cell is an immune cell.

147. The method of any one of claims 116-146, wherein the immune cell is a T cell and the engineered cell is an engineered T cell.

148. The method of claim 147, wherein the T cell is a CD8⁺ T cell, CD4+ T cell, or γδ T cell and the engineered cell is an engineered T cell.

149. The method of any one of claims 116-148, wherein the starting population of immune effector cells is obtained from a subject having cancer and the host cell is allogeneic or autologous to the subject.

150. The method of any one of claims 145-149, wherein a population of CD8 or CD4-positive and peptide MHC tetramer-positive engineered T cells are purified from the transduced host cells.

151. The method of any one of claims 116-150, wherein a clonal population of peptide-specific engineered T cells are generated by limiting or serial dilution followed by expansion of individual clones by a rapid expansion protocol.

152. A method of cloning a T cell receptor (TCR), the method comprising
(a) contacting a starting population of immune effector cells with the peptide or polypeptide of any one of claims 89-105, thereby generating peptide-specific immune effector cells;
(b) purifying immune effector cells specific to the peptide,
(c) isolating a TCR sequence from the purified immune effector cells.

153. The method of claim 152, wherein contacting is further defined as co-culturing the starting population of immune effector cells with antigen presenting cells (APCs), wherein the APCs present the peptide on their surface.

154. The method of claim 153, wherein the APCs are dendritic cells.

155. The method of any one of claims 152-154, wherein the immune effector cells are T cells, peripheral blood lymphocytes, NK cells, invariant NK cells, NKT cells.

156. The method of any one of claims 152-155, wherein the immune effector cells have been differentiated from mesenchymal stem cell (MSC) or induced pluripotent stem (iPS) cells.

157. The method of claim 155 or 156, wherein the T cells are CD8⁺ T cells, CD4⁺ T cells, or γδ T cells.

158. The method of any one of claims 155-157, wherein the T cells are cytotoxic T lymphocytes (CTLs).

159. The method of any one of claims 152-158, wherein the method comprises isolating the starting population of immune effector cells from peripheral blood mononuclear cells (PBMCs).

160. The method of any of claims 152-159, wherein the starting population of immune effector cells is obtained from a subject.

161. The method of claim 160, wherein the subject is a human.

162. The method of claim 160 or 161, wherein the subject has cancer.

163. The method of claim 162, wherein the cancer comprises a TBXT antigen positive cancer.

164. The method of claim 163, wherein the cancer comprises cancer cells that are positive for a peptide of SEQ ID NO: 15.

165. The method of any one of claims 160-164, wherein the subject has been determined to have a TBXT antigen positive cancer.

166. The method of claim 165, wherein the subject has been determined to have cancer cells that are positive for a peptide of SEQ ID NO: 15.

167. The method of any one of claims 162-166, wherein the cancer comprises chordoma.

168. The method of any one of claims 162-166, wherein the cancer comprises small cell lung cancer.

169. The method of any one of claims 152-168, wherein the method further comprises introducing the peptide or a nucleic acid encoding the peptide into the dendritic cells prior to the co-culturing.

170. The method of claim 169, where the peptide or nucleic acid encoding the peptide are introduced by electroporation.

171. The method of claim 169, wherein the peptide or nucleic acid encoding the peptide are introduced by adding the peptide or nucleic acid encoding the peptide to the media of the dendritic cells.

172. The method of any one of claims 154-171, wherein the immune effector cells are co-cultured with a second population of dendritic cells into which the peptide or a nucleic acid encoding the peptide has been introduced.

173. The method of any one of claims 152-172, wherein purifying is defined as purifying a population of CD8-positive and peptide MHC tetramer-positive T cells from the immune effector cells following the co-culturing.

174. The method of claim 173, wherein the population of CD8-positive and peptide MHC tetramer-positive T cells are purified by fluorescence activated cell sorting (FACS).

175. The method of claim 174, wherein purifying further comprises generation of a clonal population of peptide-specific immune effector cells by limiting or serial dilution of sorted cells followed by expansion of individual clones by a rapid expansion protocol.

176. The method of claim 175, wherein the method further comprises cloning of a T cell receptor (TCR) from the clonal population of peptide-specific immune effector cells.

177. The method of any one of claims 152-176, wherein the method further comprises sequencing the TCR alpha and/or beta gene(s) and/or performing grouping of lymphocyte interactions by paratope hotspots (GLIPH) analysis.

178. The method of claim 176 or 177, wherein cloning of the TCR is cloning of a TCR alpha and a beta chain.

179. The method of claim 178, wherein the TCR alpha and beta chains are cloned using a 5′-Rapid amplification of cDNA ends (RACE) method.

180. The method of claim 179, wherein the cloned TCR is subcloned into an expression vector.

181. The method of claim 180, wherein the expression vector comprises a linker domain between the TCR alpha sequence and TCR beta sequence.

182. The method of claim 181, wherein the linker domain comprises a sequence encoding one or more peptide cleavage sites.

183. The method of claim 182, wherein the one or more cleavage sites are a Furin cleavage site and/or a P2A cleavage site.

184. The method of claim 183, wherein the TCR alpha sequence and TCR beta sequence are linked by an IRES sequence.

185. The method of any of claims 180-184, wherein the expression vector is a retroviral or lentiviral vector.

186. The method of claim 185, where a host cell is transduced with the expression vector to generate an engineered cell that expresses the TCR alpha and beta chains.

187. The method of claim 186, wherein the host cell is an immune cell.

188. A peptide-specific engineered T cell produced according to any one of the methods of claims 116-187.

189. A TCR produced by the method of any one of claims 152-185.

190. A fusion protein comprising the TCR of any one of claim 27-44, 71-88, or 189 and a CD3 binding region.

191. The fusion protein of claim 190, wherein the CD3 binding region comprises a CD3-specific fragment antigen binding (Fab), single chain variable fragment (scFv), single domain antibody, or single chain antibody.

192. The TCR of any one of claims 27-44 and 71-88 or the fusion protein of claim 190 or 191, wherein the TCR or fusion protein is conjugated to a detection or therapeutic agent.

193. The TCR or fusion protein of claim 192, wherein the agent comprises a fluorescent molecule, radiative molecule, or toxin.

194. A nucleic acid encoding the polypeptide of any one of claim 1-26, 45-70, or 105, the TCR of any one of claim 27-44, 71-88, 189, 192, or 193, the peptide of any one of claims 89-104 or the fusion protein of any one of claims 190-193.

195. The nucleic acid of claim 194, wherein the nucleic acid is RNA.

196. The nucleic acid of claim 194, wherein the nucleic acid is DNA or a cDNA encoding the peptide or polypeptide or a complement of the peptide or polypeptide.

197. The nucleic acid of claim 194, wherein the nucleic acid has at least 70% sequence identity to one of SEQ ID NOS: 1, 2, 28, 29 or a fragment thereof.

198. A nucleic acid expression vector comprising the nucleic acid(s) of any one of claims 194-197.

199. The vector of claim 198, wherein the vector comprises a promoter that directs the expression of the nucleic acid.

200. The vector of claim 199, wherein the promoter comprises a murine stem cell virus (MSCV) promoter.

201. The vector of any one of claims 198-200, wherein the vector comprises the TCR-a and TCR-b genes.

202. A cell comprising the polypeptide of any one of claim 1-26, 45-70, or 105, the TCR of any one of claim 27-44, 71-88, 189, 192, or 193, the fusion protein of any one of claims 190-193, the nucleic acid(s) of any one of claims 194-197, or the vector of any one of claims 198-201.

203. The cell of claim 202, wherein the cell comprises a stem cell, a progenitor cell, an immune cell, or a natural killer (NK) cell.

204. The cell of claim 203, wherein the cell comprises a hematopoietic stem or progenitor cell, a T cell, a cell differentiated from mesenchymal stem cells (MSCs) or an induced pluripotent stem cell (iPSC).

205. The cell of claim 203 or 204, wherein the cell is isolated or derived from peripheral blood mononuclear cell (PBMCs).

206. The cell of claim 204 or 205, wherein the T cell comprises a cytotoxic T lymphocyte (CTL), a CD8⁺ T cell, a CD4⁺ T cell, an invariant NK T (INKT) cell, a gamma-delta T cell, a NKT cell, or a regulatory T cell.

207. The cell of any one of claims 202-206, wherein the cell is isolated from a cancer patient.

208. An in vitro isolated dendritic cell comprising the peptide or polypeptide of any one of claims 89-105, the nucleic acid of any one of claims 194-197, or the vector of any one of claims 198-201.

209. The dendritic cell of claim 208, wherein the dendritic cell is a mature dendritic cell.

210. The dendritic cell of claim 208 or 209, wherein the cell is a cell with an HLA-A, HLA-B, or HLA-C type.

211. The dendritic cell of claim 210, wherein the cell is a HLA-A, HLA-A02, or a HLA-A0201 type.

212. A composition comprising the polypeptide of any one of claim 1-26, 45-70, or 105, the TCR of any one of claim 27-44, 71-88, 189, 192, or 193, the fusion protein of any one of claims 190-193, the nucleic acid(s) of any one of claims 194-197, the vector of any one of claims 198-201, or the cell of any one of claims 202-211.

213. The composition of claim 212, wherein the composition is formulated for parenteral administration, intravenous injection, intramuscular injection, inhalation, or subcutaneous injection.

214. The composition of claim 212 or 213, wherein the peptide is comprised in a liposome, lipid-containing nanoparticle, or in a lipid-based carrier.

215. The composition of any one of claims 212-214, wherein the composition is formulated as a vaccine.

216. The composition of any one of claims 212-215, wherein the composition further comprises an adjuvant.

217. The composition of any one of claims 212-216, wherein the composition has been determined to be serum-free, mycoplasma-free, endotoxin-free, and/or sterile.

218. A method of making an engineered cell comprising the nucleic acid(s) of any one of claims 194-197 or the vector of any one of claims 198-201 into a cell.

219. The method of claim 218, wherein the method further comprises culturing the cell in media, incubating the cell at conditions that allow for the division of the cell, screening the cell, and/or freezing the cell.

220. A method for treating cancer in a subject comprising administering the composition of any one of claims 212-217 or the cell of any one of claim 188 or 202-211 to a subject in need thereof.

221. A method for treating or preventing cancer in a subject comprising administering the composition of any one of claims 212-217 or the cell of any one of claim 188 or 202-211 to a subject in need thereof.

222. The method of claim 220 or 221, wherein the cancer is chordoma.

223. The method of claim 220 or 221, wherein the cancer is small cell lung cancer.

224. A method of stimulating an immune response in a subject, the method comprising administering the composition of any one of claims 212-217 or the cells of any one of claim 188 or 202-211 to a subject in need thereof.

225. The method of any one of claims 220-224, wherein the subject is a human subject.

226. The method of any one of claims 220-225, wherein the cells are autologous.

227. The method of any one of claims 220-225, wherein the cells are allogenic.

228. The method of any one of claims 220-227, wherein the subject has previously been treated for the cancer.

229. The method of claim 228, wherein the subject has been determined to be resistant to the previous treatment.

230. The method of any one of claims 220-229, wherein the method further comprises the administration of an additional therapy.

231. The method of any one of claims 220-230, wherein the cancer comprises stage I, II, III, or IV cancer.

232. The method of any one of claims 220-231, wherein the cancer comprises metastatic and/or recurrent cancer.

233. The method of any one of claims 220-232, wherein the cancer is a TBXT antigen positive cancer.

234. The method of any one of claims 220-233, wherein the subject has been determined to have TBXT antigen positive cancer cells.

235. The method of any one of claims 220-234, wherein the subject has been determined to have cancer cells that express a peptide of SEQ ID NO: 15.

236. The method of any one of claims 228-235, wherein the cancer is chordoma.

237. The method of any one of claims 228-235, wherein the cancer is small cell lung cancer.

238. The method of any one of claims 220-237, wherein the subject is and/or has been determined to be HLA-A positive.

239. The method of claim 220-238, wherein the subject is and/or has been determined to be HLA-A0201 positive.

240. A method for prognosing a patient or for detecting T cell responses in a patient, the method comprising: contacting a biological sample from the patient with the peptide or polypeptide of any one of claims 89-105 or the molecular complex of claim 113.

241. The method of claim 240, wherein the biological sample comprises a blood sample or a fraction thereof.

242. The method of claim 241, wherein the biological sample comprises lymphocytes.

243. The method of claim 242, wherein the biological sample comprises a fractionated sample comprising lymphocytes.

244. The method of any one of claims 240-243, wherein the peptide is linked to a solid support.

245. The method of claim 244, wherein the peptide is conjugated to the solid support or is bound to an antibody that is conjugated to the solid support.

246. The method of claim 244 or 245, wherein the solid support comprises a microplate, a bead, a glass surface, a slide, or a cell culture dish.

247. The method of any one of claims 240-246, wherein detecting T cell responses comprises detecting the binding of the peptide to the T cell or TCR.

248. A kit comprising the peptide or polypeptide of any one of claims 89-105 in a container.

249. The kit of claim 248, wherein the peptide is comprised in a pharmaceutical preparation.

250. The kit of claim 249, wherein the pharmaceutical preparation is formulated for parenteral administration or inhalation.

251. The kit of claim 248, wherein the peptide is comprised in a cell culture media.
